(19) United States
(12) Patent Application Publication
(10) Pub. No.: US 2026/0224476 A1
(43) Pub. Date: Aug. 6, 2026

(54) ISOXAZOLINE PARASITICIDE FORMULATIONS AND METHODS FOR TREATING BLEPHARITIS

(71) Applicant: Tarsus Pharmaceuticals, Inc., Irvine, CA (US)

(72) Inventors: Bobak Robert Azamian, Newport Coast, CA (US); Seshadri Neervannan, Irvine, CA (US); Bryan Wahl, Irvine, CA (US)

(21) Appl. No.: 19/154,760

(22) PCT Filed: Feb. 8, 2024
(86) PCT No.: PCT/US2024/015000

§ 371 Date: Aug. 7, 2025

Related U.S. Application Data
(60) Provisional application No. 63/484,435, filed on Feb. 10, 2023.

(51) Int. Cl. A61K 9/00; A61K 31/422; A61K 47/10; A61K 47/26; A61K 47/38; A61P 27/02

(52) U.S. Cl. CPC A61K 9/0048; A61K 31/422; A61K 47/10; A61K 47/26; A61K 47/38; A61P 27/02

(57) ABSTRACT

Disclosed herein are methods for treating or preventing ophthalmic and dermatologic conditions in a patient, including ocular surface conditions such as blepharitis. The methods can include topically administering directly to an ocular surface of one or more eyes of a patient in need of treatment thereof an effective amount of an isoxazoline parasiticide, formulated into an ophthalmic composition, the ophthalmic composition further comprising a pharmaceutically acceptable vehicle. Compositions are also disclosed.

### PRIORITY CLAIM

This application claims the benefit under 35 U.S.C. § 119 (e) of U.S. Prov. App. No. 63/484,435 filed on Feb. 10, 2023, which is hereby incorporated by reference in its entirety.

### BACKGROUND

Blepharitis, or inflammation of the eyelids, is a common, often chronic condition that can be challenging to treat, and affect any age group. Blepharitis can be both anterior (outer surface of the eyelid affected, where the eyelashes are attached) and/or posterior (inner surface of the eyelid affected). Blepharitis can be associated with systemic diseases including rosacea and seborrheic dermatitis, and be related to other ophthalmic diseases including chalazion, conjunctivitis, keratitis, and dry eyes.

Demodex folliculorum and Demodex brevis are microscopic, obligate, elongated mites that are the most common permanent intracutaneous parasites inhabiting the hair follicles and sebaceous glands of humans and animals. A total of 65 Demodex species have been described, parasitizing 11 orders of mammals and belonging to the family Demodicidae of the order Acarina, class Arachnida. Mating takes place in the follicle opening and eggs are laid inside the hair follicles or sebaceous glands. The six-legged larvae hatch after 3-4 days, and the larvae develop into adults in about 7 days. Demodex has a life cycle of about 14 days. The total lifespan of a Demodex mite is several weeks. The dead mites decompose inside the hair follicles or sebaceous glands.

Demodex can be found on the face, including cheeks, nose, chin, forehead, temples, eye lashes, brows, and also on the scalp, neck, and ears. Other seborrheic locations such as nasolabial folds, peri-orbital areas, and less commonly upper and medial region of chest and back can also be infested. Demodex may also be found on the penis, mons veneris, buttocks, and in the ectopic sebaceous glands in the buccal mucosa. In some cases, a mite density of greater than 5 mites/cm2 in the pilosebaceous unit or 5 or more mites per follicle correlates with a Demodex infestation.

Demodex infestation can be diagnosed clinically be removing lashes (epilating), with a mite density greater than 1 mite per lash generally considered to be positive. Another specific sign that is pathognomonic (80%+ correlation) for Demodex infestation is the presence of collarettes (aka cylindrical dandruff), the result of mite byproducts. This sign can be detected with a slit lamp examination or other magnified examination of the eyelid margin and can aid clinicians in diagnosing Demodex blepharitis. Both collarettes and mite density can also be used as clinical inclusion criteria and endpoints in clinical trials.

Blepharitis is a disease characterized by inflammation of the eyelid margins. Participants with blepharitis often experience lid margin itching, burning, or stinging in the eyes, foreign body sensation, as well as redness of the eyes and eyelids. In the United States, blepharitis is estimated to affect as many as 19 million people and is most common in individuals over the age of 50. One of the most common causes of blepharitis can be attributed to Demodex mites.

Demodex mites (Demodex folliculorum or Demodex brevis) are microscopic, obligate ectoparasites that inhabit the base of eyelashes, Meibomian glands, and sebaceous glands. Demodex are the only mites that are known to affect the human eye. A recent meta-analysis of 11 clinical studies has shown that Demodex is associated with 45% of blepharitis cases. The density of Demodex is correlated with the signs of blepharitis and other ocular complications.

In Demodex blepharitis, mites inhabit the lash follicles where they feed on sebum and cause epithelial abrasions, resulting in epithelial hyperplasia and reactive hyperkeratinization. Undigested material, combined with epithelial cells, keratin, and eggs lead to the formation of collarettes (also known as cylindrical dandruff), which are pathognomonic for Demodex blepharitis. Lashes with collarettes have been demonstrated to contain 2-4 mites/lash on average, compared with only 0.2 mites/lash without collarettes. Demodex infestations can also lead to swelling and irritation, as well as enlargement of the Meibomian glands. A localized granulomatous reaction associated with the chitinous exoskeleton and waste products of Demodex may mechanically block the Meibomian glands. In addition to surface irritation and inflammation, the mites may also activate inflammatory cascades by introducing bacteria harbored on and within the mites. Finally, the lid collarettes caused by Demodex infestation can themselves trigger an immune response and have been shown to increase the number of CD4+ T cells, macrophages, and Langerhans in participants.

Among a wide range of reported species, only two, Demodex folliculorum and Demodex brevis, are found to parasitize the human body surface. Demodex folliculorum has been found, for example, on eyelash follicles, while Demodex brevis has been found, for example, proximate Meibomian (tarsal) glands around the eye and sebaceous glands of the skin. Meibomian glands are a holocrine type of exocrine glands, at the rim of the eyelids inside the tarsal plate, responsible for the supply of meibum, an oily substance that prevents evaporation of the eye's tear film. Meibum prevents tear spillage onto the cheek, trapping tears between the oiled edge and the eyeball, and makes the closed lids airtight. There are approximately 50 glands on the upper eyelids and 25 glands on the lower eyelids. Symbiotic bacteria on the mites also can contribute to pathology. Increased sebum secretion and an increased number of sebaceous glands can provide a favorable habitat for the mites. While some level of Demodex can be asymptomatic, multiplication of Demodex mites to high densities, and/or a concurrent immune imbalance usually leads to skin damage. A growing body of literature implicates Demodex mites in anterior and posterior blepharitis. For example, Demodex has been implicated in 45% of blepharitis cases. It has been estimated that the prevalence of ocular surface disease is about 30 million patients; 19 million of which have Meibomian gland dysfunction/posterior blepharitis; 9 million with Demodex infestation, and 4 million with clear signs of Demodex. Blepharitis is a significant diagnosis, with no approved therapy currently in the US. Safe, efficacious therapies to treat blepharitis and other ophthalmic and dermatologic conditions are needed.

### SUMMARY

In some embodiments, disclosed herein are topical therapeutic agents, including but not limited to topical pharmaceutical agents including one or more isoxazoline parasiticide, for the treatment of various ophthalmic and dermatologic conditions.

Not to be limited by theory, the inventors have discovered formulations and methods for treating Demodex that are unexpectedly effective and durable with a minimal side effect profile. Some embodiments of formulations are unexpectedly beneficial in that they include aqueous solubility and specific excipients suitable to an eye drop (including but not limited to single-dose eye drop formulations and multiple-dose eye drop formulations); arc within specific ranges of drug concentration for local effects without leading to significant systemic drug levels (e.g., below a therapeutic concentration range); and result in rapid, complete, and durable responses never before seen in blepharitis.

In some embodiments, a formulation can include any number of combinations of features described in this disclosure.

In some embodiments, a device can include any number of combinations of features described in this disclosure.

In some embodiments, a method can include any number of combinations of features described in this disclosure.

In some configurations, disclosed herein is a method for treating blepharitis in a patient, including topically administering directly to an ocular surface of one or more eyes of a patient in need of treatment thereof an effective amount of an isoxazoline parasiticide, formulated into an ophthalmic composition, the ophthalmic composition including a pharmaceutically acceptable vehicle.

In some configurations, the ophthalmic composition can be sterile and non-irritating to the eye.

In some configurations, the isoxazoline parasiticide can be the sole active ingredient of the ophthalmic composition.

In some configurations, from about 0.01% to about 1% of the isoxazoline parasiticide with respect to the total weight of the composition can be administered.

In some configurations, about 0.03% by weight of the isoxazoline parasiticide with respect to the total weight of the composition can be administered.

In some configurations, about 0.10% by weight of the isoxazoline parasiticide with respect to the total weight of the composition can be administered.

In some configurations, about 0.30% by weight of the isoxazoline parasiticide with respect to the total weight of the composition can be administered.

In some configurations, the ophthalmic composition can include an eye drop.

In some configurations, the ophthalmic composition can not include any essential oils.

In some configurations, the isoxazoline parasiticide can be selected from the group consisting of: fluralaner, sarolaner, lotilaner, afoxolaner, fluxametamide, isocycloseram, mivorilaner, umifoxolaner, esafoxolaner, tigolaner, and modoflaner.

In some configurations, the ocular surface can include at least one of the conjunctiva or cornea of the one or more eyes of the patient.

In some configurations, the ophthalmic composition can include a polysorbate or castor oil solubilizing agent. As described herein, a castor oil solubilizing agent can include but is not limited to castor oil or a derivative thereof, such as, e.g., hydrogenated castor oil, PEG-35 castor oil, PEG-35 hydrogenated castor oil, polyoxyl 35 castor oil (Cremophor EL), polyoxyl 40 hydrogenated castor oil (Cremophor RH 40), polyoxyl 40 stearate, or Cremophor RH 60 (polyoxyl 60 hydrogenated castor oil).

In some configurations, the formulation can include a polymer to enhance viscosity (e.g., HPMC, CMC), a sugar for physiologic osmolality (e.g., glycerin, sorbitol, mannitol, or others), a buffer for neutral to slightly basic pH (e.g., a phosphate, TRIS), and a preservative (e.g., LAK or BAK, or a sorbate).

In some configurations, disclosed herein is a method for treating blepharitis in a patient, including topically administering directly to one or more of the eye, eyelids, or eyelashes of a patient in need of treatment thereof an effective amount of an isoxazoline parasiticide, formulated into an ophthalmic composition including a pharmaceutically acceptable vehicle, wherein the ophthalmic composition is sterile and non-irritating to the eye, and wherein the isoxazoline parasiticide is the sole active ingredient of the ophthalmic composition.

In some configurations, the patient's eyes can be closed upon topically administering the ophthalmic composition, such that the composition contacts orifices of Meibomian glands of the patient and outside of eyelid margins of the patient.

In some configurations, the method can include spreading the composition onto the eyelashes and follicles of the eyelashes.

In some configurations, the method can include spreading the composition onto the eyelashes and follicles of the eyelashes with an applicator.

In some configurations, from about 0.001% to about 1% of the isoxazoline parasiticide can be administered.

In some configurations, the method can include topically administering the ophthalmic composition at least once daily for at least about 2 weeks.

In some configurations, the method can include topically administering the ophthalmic composition at least once daily for about or at least about 4 weeks or 6 weeks.

In some configurations, disclosed herein is a method for treating an ocular Demodex infestation in a patient, including topically administering directly to one or more of the eyes, eyelids, or eyelashes of one or more eyes of a patient in need of treatment thereof, an effective amount of an isoxazoline parasiticide, formulated into an ophthalmic composition further comprising a pharmaceutically acceptable vehicle, wherein the ophthalmic composition is sterile and non-irritating to the eye, and wherein the isoxazoline parasiticide is the sole active ingredient of the ophthalmic composition.

In some configurations, the method can include receiving a first assessment of a quantity of Demodex mites or collarettes on an anatomical structure of the patient, and topically administering the ophthalmic composition if the quantity of Demodex mites or collarettes is greater than a predetermined value.

In some configurations, the ophthalmic formulation can cause an abdomen and tail of Demodex mites on the patient to stop moving more quickly relative to a cephalothorax of the Demodex mites.

In some configurations, disclosed herein is a method of treating blepharitis and/or rosacea, including topically applying isoxazoline parasiticide proximate one or more eyelashes, the topically applying therapeutically effective to preferentially be absorbed by a body of the Demodex mite with respect to ingestion by the Demodex mite sufficient to cause reduced movement of the body of the Demodex mite with respect to a head of the Demodex mite, the method sufficient to reduce or eliminate Demodex mites proximate the eyelashes, resulting in improvement of the manifestations of blepharitis and/or rosacea.

In some configurations, disclosed herein is a topical ophthalmic formulation for treating blepharitis in a patient, including an effective amount of an isoxazoline parasiticide and a pharmaceutically acceptable vehicle, wherein the ophthalmic composition is sterile and non-irritating to the eye, and wherein the isoxazoline parasiticide is the sole active ingredient of the ophthalmic composition.

In some configurations, from about 0.01% to about 1% of the isoxazoline parasiticide with respect to the total weight of the composition can be administered.

In some configurations, about 0.03% by weight of the isoxazoline parasiticide with respect to the total weight of the composition can be administered.

In some configurations, about 0.10% or 0.3% by weight of the isoxazoline parasiticide with respect to the total weight of the composition can be administered.

In some configurations, the ophthalmic composition can include an eye drop.

In some configurations, the ophthalmic composition can not include any essential oils.

In some configurations, the isoxazoline parasiticide can be selected from the group consisting of: fluralaner, sarolaner, lotilaner, afoxolaner, fluxametamide, isocycloseram, mivorilaner, umifoxolaner, esafoxolaner, tigolaner, and modoflaner.

In some configurations, disclosed herein is a topical formulation for use in treating an ocular surface disease, including an isoxazoline parasiticide, at least one of Pemulen and HPMC, polysorbate 80 or castor oil solubilizing agent, glycerin, a buffering agent, and lauralkonium or benzalkonium chloride, wherein the formulation is therapeutically effective to reduce or eliminate Demodex mites proximate the eyelashes, resulting in improvement of the manifestations of blepharitis and/or rosacea. In some configurations, the formulation cannot include a polysorbate and/or a phosphate buffering agent.

In some configurations, the formulation can be for use in treating blepharitis.

In some configurations, the formulation can be for use in treating anterior blepharitis.

In some configurations, the formulation can be for use in treating posterior blepharitis, Meibomian gland dysfunction, or dry eye disease.

In some configurations, the formulation can be for use in treating ocular rosacea including corneal inflammation, ulceration, or scarring.

In some configurations, disclosed herein is a method for treating lice, scabies, and/or bed bugs in a patient, including topically administering directly to a skin surface of a patient in need of treatment thereof an effective amount of an isoxazoline parasiticide, formulated into a composition, the composition further comprising a pharmaceutically acceptable vehicle. The composition can be non-irritating to the skin. The isoxazoline parasiticide can be the sole active ingredient of the composition, or in combination with other active ingredients.

In some configurations, disclosed herein is a method for preventing vector-borne disease in a patient, including topically administering directly to a skin surface of a patient in need of treatment thereof an effective amount of an isoxazoline parasiticide, formulated into a composition, the composition further comprising a pharmaceutically acceptable vehicle. The composition can be non-irritating to the skin. The isoxazoline parasiticide can be the sole active ingredient of the composition, or in combination with other active ingredients.

In some configurations, disclosed herein is a method for improving eyelash health in a patient. The method can include topically administering directly to an ocular surface of one or more eyes of a patient in need of treatment thereof an effective amount of an isoxazoline parasiticide, formulated into an ophthalmic composition, the ophthalmic composition further comprising a pharmaceutically acceptable vehicle. The ophthalmic composition can be sterile and non-irritating to the eye. The isoxazoline parasiticide can be the sole active ingredient of the composition, or in combination with other active ingredients. Improving eyelash health can increase, for example, the number, growth rate, thickness, and/or appearance of eyelashes of the patient.

In some configurations, the composition is not systemically absorbed, or not systemically absorbed sufficient to have a therapeutic effect or any adverse effects systemically.

In some embodiments, disclosed herein is a topical, multi-dose ophthalmic or dermatologic formulation for use in treating an ocular surface disease. The formulation can include any number of: an isoxazoline parasiticide; a castor oil solubilizing agent; a polyol; an acid; tromethamine (TRIS); and lauralkonium or benzalkonium chloride. The formulation can be therapeutically effective to reduce or eliminate Demodex mites proximate the eyelashes, resulting in improvement of the manifestations of blepharitis and/or rosacca.

In some configurations, the topical formulation is for use in treating blepharitis (e.g., anterior or posterior blepharitis). In some configurations, the topical formulation is for use in treating ocular rosacea.

In some configurations, the isoxazoline parasiticide comprises between about 0.01% and about 0.50% w/w of the formulation, such as about 0.01%, 0.05%, 0.10%, 0.15%, 0.20%, 0.25%, 0.30%, 0.35%, 0.40%, 0.45%, 0.50%, or ranges including any two of the foregoing values.

In some configurations, the ophthalmic composition does not include any essential oils.

In some configurations, an isoxazoline parasiticide is selected from the group consisting of: fluralaner, sarolaner, lotilaner, afoxolaner, fluxametamide, isocycloseram, mivorilaner, umifoxolaner, esafoxolaner, tigolaner, and modoflaner.

In some configurations, a castor oil solubilizing agent comprises between about 3% and about 7% w/w of the formulation, or about 5% w/w of the formulation.

In some configurations, a polyol is selected from the group consisting of glycerol, xylitol, erythritol, mannitol, and sorbitol.

In some configurations, a formulation further comprises HPMC or CMC.

In some configurations, a formulation does not comprise a phosphate buffer.

In some configurations, a formulation does not comprise a polysorbate.

In some configurations, disclosed herein is a topical gel formulation for ophthalmic or dermatologic use, including an isoxazoline parasiticide, and any number of the following features, or others disclosed herein: a castor oil solubilizing agent; HPMC, a carbomer; tromethamine (TRIS), a polyol; and/or a preservative, (e.g., lauralkonium chloride and benzalkonium chloride).

In some configurations, disclosed herein is a single-dose ophthalmic formulation for use in treating an ocular surface disease, including any number of: an isoxazoline parasiticide; a castor oil solubilizing agent; a polyol; and tromethamine (TRIS), wherein the formulation does not comprise a preservative agent, wherein the formulation is therapeutically effective to reduce or eliminate Demodex mites proximate the eyelashes, resulting in improvement of the manifestations of blepharitis and/or rosacea.

In some configurations, a formulation can include one or more of a lipid-producing agent, keratolytic agent, and/or a mucolytic agent. In some embodiments, disclosed herein are topical, multi-dose ophthalmic formulations for use in treating an ocular surface disease, comprising: an isoxazoline parasiticide; a castor oil solubilizing agent; a viscosity-enhancing agent; a buffering agent; a sorbate; and/or a chelating agent. In some embodiments, the formulation is therapeutically effective to reduce or eliminate Demodex mites proximate the eyelashes, resulting in improvement of the manifestations of blepharitis and/or rosacea.

In some embodiments, the sorbate comprises potassium sorbate, the chelating agent comprises EDTA, and the viscosity-enhancing agent comprises HPMC. In some embodiments, the pH of the formulation is between about 7 and about 7.5. In some embodiments, the pH of the formulation is about 7.2.

In some configurations, disclosed herein is a method of for treating blepharitis in a patient, said method comprising topically administering directly to an ocular surface of one or more eyes of a patient in need of treatment thereof an effective amount of an isoxazoline parasiticide, formulated into an ophthalmic composition with sorbate and a pharmaceutically acceptable vehicle, said ophthalmic composition being sterile and non-irritating to the eye. The isoxazoline parasiticide can be the sole active ingredient of the ophthalmic composition. In some embodiments, the sorbate is the only preservative agent of the composition.

In some configurations, disclosed herein is a method for treating blepharitis in a patient, said method comprising topically administering directly to one or more of the eye, eyelids, or eyelashes of a patient in need of treatment thereof an effective amount of an isoxazoline parasiticide, formulated into an ophthalmic composition with a sorbate, a castor oil solubilizing agent, and a pharmaceutically acceptable vehicle, said ophthalmic composition being sterile and non-irritating to the eye. The isoxazoline parasiticide can be the sole active ingredient of the ophthalmic composition.

In some configurations, disclosed herein is a method for treating an ocular Demodex infestation in a patient, said method comprising topically administering directly to one or more of the eyes, eyelids, or eyelashes of one or more eyes of a patient in need of treatment thereof, an effective amount of an isoxazoline parasiticide, formulated into an ophthalmic composition with a sorbate and a pharmaceutically acceptable vehicle, said ophthalmic composition being sterile and non-irritating to the eye. The isoxazoline parasiticide can be the sole active ingredient of the ophthalmic composition. A volume of the composition delivered can be less than about 100 microliters.

In some configurations, disclosed herein is a method of treating blepharitis and/or rosacea, said method comprising topically applying a composition comprising isoxazoline parasiticide proximate one or more eyelashes, the topically applying therapeutically effective to preferentially be absorbed by a body of the Demodex mite with respect to ingestion by the Demodex mite sufficient to cause reduced movement of the body of the Demodex mite with respect to a head of the Demodex mite, the method sufficient to reduce or eliminate Demodex mites proximate the eyelashes, resulting in improvement of the manifestations of blepharitis and/or rosacea. The composition can further comprise a sorbate and a castor oil solubilizing agent, wherein the sorbate is the sole preservative agent of the composition.

In some configurations, disclosed herein is a topical ophthalmic formulation for treating blepharitis in a patient, said formulation comprising a sorbate, a castor oil solubilizing agent and an effective amount of an isoxazoline parasiticide and a pharmaceutically acceptable vehicle, said ophthalmic composition being sterile and non-irritating to the eye. The isoxazoline parasiticide can be the sole active ingredient of the ophthalmic composition. The sorbate can be the sole preservative agent of the composition.

In some configurations, disclosed herein is a topical formulation for use in treating an ocular surface disease, said formulation comprises an isoxazoline parasiticide; at least one of Pemulen and HPMC; castor oil solubilizing agent; glycerin; a buffering agent; and potassium sorbate. The formulation can be therapeutically effective to reduce or eliminate Demodex mites proximate the eyelashes, resulting in improvement of the manifestations of blepharitis and/or rosacea.

In some configurations, disclosed herein is a topical, ophthalmic formulation for use in treating an ocular surface disease, said formulation comprising an isoxazoline parasiticide; a castor oil solubilizing agent; and a polyol, said formulation being therapeutically effective to reduce or eliminate Demodex mites proximate the eyelashes, resulting in improvement of the manifestations of blepharitis and/or rosacca.

### BRIEF DESCRIPTION OF THE FIGURES

FIGS. 1A-B schematically illustrate application of an ophthalmic formulation onto an eye with a Demodex infestation.

FIG. 2 illustrates data illustrating activity of selected anatomy of Demodex mites following therapy with a topical formulation.

FIGS. 3A-3B show examples of formulations with amitraz, fluralaner, and lotilaner.

FIGS. 4A-4C illustrate embodiments of various diagnostic techniques for Demodex that do not necessarily require epilation, including examination of collarettes.

FIGS. 5A-5C illustrate human study data relating to isoxazoline parasiticide formulations.

### DETAILED DESCRIPTION

In some embodiments, disclosed herein are topical therapeutic agents, including but not limited to topical pharmaceutical agents including one or more isoxazoline parasiticide, for the treatment of various ophthalmic and dermatologic conditions. Also disclosed herein are methods of treating blepharitis, ocular rosacea, and Demodex infestation in patients in need thereof. In some embodiments, patients in need thereof can be treated with an active agent from the isoxazoline parasiticide family of chemicals, which include but are not limited to isoxazoline-substituted benzamide derivatives. Not to be limited by theory, isoxazoline parasiticides can act as a GABA-chloride antagonist to selectively target the nervous system of certain organisms, including but not limited to Demodex. The GABA-mediated chloride influx can lead to hyperpolarization of the cellular membrane and generates an inhibitory postsynaptic potential, which decreases the probability of an action potential, and lead to paralysis and eventual death of Demodex mites. The isoxazoline parasiticide can include, for example, any number of fluralaner, sarolaner, lotilaner, afoxolaner, fluxametamide, isocycloseram, mivorilaner, umifoxolaner, esafoxolaner, tigolaner, and/or modoflaner, including derivatives, analogues, and L- and D-isomers thereof, including but not limited to enantiomers, compositions comprising racemic mixtures, and enantiomerically pure compositions. In some embodiments, the isoxazoline parasiticide is the only active ingredient utilized in the formulation and/or method. In some embodiments, the isoxazoline parasiticide is an isoxazoline-substituted benzamide derivative. In some embodiments, the isoxazoline parasiticide has one, two, three, or more fluorine groups, such as trifluorine groups in its chemical structure (e.g., R—CF₃). In some embodiments, the formulation can include a precursor compound (e.g., an isoxazole carboxylic acid, including isoxazole-4-carboxylic acid), or a degradation compound (e.g., isoxazolethiopene carboxylic acid) to other isoxazoline parasiticides, instead of or in addition to isoxazoline parasiticides disclosed elsewhere herein, in amounts/concentrations disclosed elsewhere herein, such as between about 0.001% to about 0.1%, or between about 0.005% to about 0.01%, for example. In some embodiments, a formulation does not include any precursor or degradation compounds, including those disclosed herein. In some embodiments, a formulation can include a pyrazole-5-carboxamides including an arylisoxazoline moiety.

Isoxazoline parasiticides have been conventionally utilized for veterinary applications, including chews and non-ophthalmic topical “pour on” solutions, although to the inventors' knowledge no human formulations have been developed. Non-limiting examples of isoxazoline parasiticides can be found, for example, in U.S. Pat. No. 7,662,972 to Mita et al., U.S. Pat. No. 8,466,115 to Curtis et al., U.S. Pat. No. 7,964,204 to Lahm et al., and U.S. Pat. No. 8,383,659 to Nanchen et al., each of which are hereby incorporated by reference in their entireties. Additionally, U.S. Pub. No. 2010/0254960 A1, PCT Pub. No. WO 2007/070606 A2, PCT Pub. No. WO 2007/123855 A2, PCT Pub. No. WO 2010/003923 A1, U.S. Pat. Nos. 7,951,828, 7,662,972, U.S. Pub. No. 2010/0137372 A1, U.S. Pub. No. 2010/0179194 A2, U.S. Pub. No. 2011/0086886 A2, U.S. Pub. No. 2011/0059988 A1, US 2010/0179195 A1, PCT Pub. No. WO 2007/075459 A2 and U.S. Pat. No. 7,951,828, all of which are incorporated by reference in their entireties, describe various other parasiticidal isoxazoline compounds. Further non-limiting examples of isoxazoline parasiticides can be found, for example, in U.S. Pub. No. 2014/0243375 to El Qacemi et al., and U.S. Pat. No. 8,735,362 to Cassayre et al., which are hereby incorporated by reference in their entireties.

Veterinary oral (e.g., non-topical) formulations such as chews result in first pass liver metabolism as well as systemic effects, which can be undesirable in some cases for targeted local applications. A significant challenge is that the fluorinated and/or chlorinated groups of certain isoxazoline parasiticides cause these molecules to be highly insoluble in any pharmaceutical-based solutions including oil and water-based solutions and having a solubility of less than about 1 mg/kg, or 1 mg/L aqueous concentrations, or even less. Veterinary topical solutions of isoxazoline parasiticide have included, for example, dimethylacetamide, glycofurol, diethyltoluamide, and/or acetone. However, such solutions only indicated as a “pour on” solution on the back of the neck of an animal, such as a cat or a dog, are unsuitable for ophthalmic use (and potentially toxic) and include instructions not to administer the solution in or around the eye. For example, the FDA posted an Animal Drug Safety Communication on Sep. 20, 2018, alerting pet owners and veterinarians to be aware of the potential for neurologic adverse events (including muscle tremors, ataxia, and seizures) in dogs and cats when treated with systemically-absorbed drugs that are in the isoxazoline class.

Such “pour on” solutions are absorbed systemically by the animal and do not result in targeted local activity only. To the inventors' knowledge, no isoxazoline parasiticide topical ophthalmic formulations have previously been developed. Therapeutic formulations that are safe and non-toxic for use, such as ocular or dermatologic use, and sufficiently soluble to be therapeutically efficacious to treat ocular Demodex and related conditions such as blepharitis, for example, are needed for both human use and for other animals.

For ophthalmic and dermatologic conditions, in some embodiments, formulations and methods are administered in an amount and/or dosage form that is not systemically bioavailable or not substantially systemically bioavailable. However, in some embodiments, formulations and methods are administered in an amount and/or dosage form that can be systemically bioavailable. In some embodiments, the formulation or method has no, or no substantial systemic or therapeutic effect outside the local targeted area, such as, for example, in or around the eye. In some embodiment, the formulation or method is not systemically absorbed or substantially systemically absorbed.

In some embodiments, the formulation or method results in a peak or random blood, plasma, serum, or other fluid level in the patient of isoxazoline parasiticide that is undetectable, or no more than about 25 ng/ml, 20 ng/ml, 15 ng/ml, or 10 ng/ml. In some embodiments, the formulation or method results in a peak or random blood, plasma, scrum, or other fluid level of isoxazoline parasiticide that is about or no more than about 5 ng/ml, 4 ng/ml, 3 ng/ml, 2 ng/ml, 1 ng/ml, 0.50 ng/ml, 0.25 ng/ml, 0.20 ng/ml, 0.15 ng/ml, 0.10 ng/ml, 0.005 ng/ml, 0.001 ng/ml, 0.0005 ng/ml, 0.0001 ng/ml or less such that the isoxazoline parasiticide has no systemic therapeutic and/or adverse effect in the patient.

It has now been determined that compounds of the family of isoxazoline parasiticide can be suitable for the treatment of ophthalmic pathologies of any origin, particularly ophthalmic pathologies due to Demodex folliculorum, and more particularly blepharitis and/or ocular rosacea. Other conditions that can be treated via formulations and methods as disclosed herein include, for example, rosacea, pityriasis folliculorum, rosacea-like demodicosis, and demodicosis gravis, nonspecific facial dermatitis, atopic dermatitis, acne vulgaris, steroid rosacea, androgenetic alopecia, madarosis, lupus miliaris disseminatus faciei, dissecting folliculitis, perioral dermatitis, acarica blepharoconjuncitivitis, papulopustular scalp eruptions, eosinophilic folliculitis, pustular folliculitis, Grover's diseases, and Demodex abscess. In some embodiments, formulations and methods including those as disclosed herein can improve eyelash health and treat madarosis, trichorrhexis, and other diseases. Not to be limited by theory, Demodex and/or other pathogens can cause inflammation and associated hair loss, lack of growth, premature depilation, and/or damage that may be associated with damage to the hair follicle and associated structures. In some embodiments, immunocompromised patients with demodicosis and be treated by systems and methods as disclosed herein. Such conditions can be surprisingly and effectively treated by formulations and methods as disclosed herein. In some embodiments, formulations and methods can have an anti-inflammatory and/or anti-microbial effect, either indirectly via eradication of Demodex and/or other parasitic organisms, and/or via a direct effect. In some embodiments, instead of or in addition to topical therapy, low-dose oral therapy of any formulation or method as disclosed herein can be utilized for maintenance therapy and/or exacerbations. The low-dose oral therapy can be, for example, a dose of 500 mg, 400 mg, 300 mg, 200 mg, 100 mg, 50 mg, 25 mg, or more or less, or ranges including any two of the foregoing values. The dose can be given, for example, daily, every other day, every third day, twice a week, once a week, every two weeks, every month, every 6 weeks, every 2 months, every 3 months, or at a longer interval.

In some embodiments, formulations and methods including those disclosed herein can be utilized for the treatment of conditions such as pediculosis (lice), including Pediculus humanus capitis (the head louse), and Pediculus humanus (the body louse). Formulations and methods can also be utilized to treat phthiriasis, such as Pthirus pubis (the crab louse or pubic louse). Infestation of the eyelashes is referred to as pediculosis ciliaris or phthiriasis palpebrarum, which can lead to intense itching of the lid margins, red watery eyes, and keratoconjunctivitis in some cases. Demodex mites are typically smaller than crab lice (0.1-0.4 mm long) and are not usually seen outside the lash follicle. Not to be limited by theory, formulations and methods such as those disclosed herein can be surprisingly effective at eliminating or inactivating lice and other organisms at low concentrations.

In some embodiments, formulations and methods including those disclosed herein can be utilized for the treatment of scabies. Scabies is an itchy skin condition caused by a tiny burrowing mite called Sarcoptes scabiei. Intense itching occurs in the area where the mite burrows. Scabies has been traditionally treated with permethrin cream, lindane lotion, crotamiton cream or lotion, or oral ivermectin. Less toxic, more effective therapies are needed.

In some embodiments, formulations and methods including those disclosed herein can be utilized for the treatment of Cimicidae, e.g., bed bugs, including Cimex lectularius.

In some embodiments, formulations and methods including those disclosed herein can be used for vector control to treat or prevent mosquito, tick, and flea-borne diseases, including but not limited to Zika virus, West Nile virus, Lyme disease, Chikungunya, Dengue fever, malaria, plague, relapsing fever, Rocky Mountain Spotted Fever, tularemia, and typhus. In some cases, application of formulations to the skin, clothing, and/or other accessories can repel, kill, inactivate, prevent or reduce the risk of bites by mosquitos, ticks, and/or fleas. In some embodiments, topical formulations are not metabolized for at least about 10, 15, 30, or 60 minutes, or 2, 3, 4, 5, 6, 7, 8, or more hours thus increasing the effective time to prevent the aforementioned bites and associated diseases.

Ivermectin is another drug that has been used to treat Demodex and is generally more soluble than isoxazoline parasiticide in solution. However, no known formulations are approved for ocular use (e.g., for blepharitis), and more efficacious therapeutic agents are needed. In some embodiments, a formulation and/or method does not involve an avermectin such as ivermectin or another macrocyclic lactone derivative. However, formulations can include an avermectin in other embodiments. In some embodiments, a formulation and/or method does not include pyrethrin, permethrin or metronidazole. However, in other embodiments, formulations can include pyrethrin, permethrin and/or metronidazole.

Disclosed herein are various embodiments of systems, methods, and formulations for the treatment of various eye conditions including but not limited to blepharitis, and the treatment of Demodex infestations (e.g., on the eyelid of a subject, such as a human). Embodiments can include any number of features as disclosed herein. Some embodiments do not include dimethylacetamide, glycofurol, diethyltoluamide, and/or acetone, at least some of which can be toxic or irritating to the eye in some cases.

Also disclosed herein is the use of a topical isoxazoline parasiticide for the treatment of blepharitis and methods of treating Demodex infestation and blepharitis in patients in need thereof.

Further disclosed is topical isoxazoline parasiticide for the treatment of rosacea and/or ocular rosacea and methods of treating Demodex infestation and rosacea and/or ocular rosacea in patients in need thereof (e.g., from the isoxazoline parasiticide family of chemicals). Formulations and methods of reducing Demodex mite count proximate the eye of the patient and cylindrical eyelash dandruff are also disclosed.

Also disclosed herein is topical isoxazoline parasiticide for the treatment of rosacea and/or ocular rosacea and methods of treating Demodex infestation and rosacea and/or ocular rosacea in patients in need thereof.

According to some embodiments, the pharmaceutical composition can include at least one, two, or more compounds selected from the family of isoxazoline parasiticide including, for example, fluralaner, sarolaner, lotilaner, afoxolaner, fluxametamide, isocycloseram, mivorilaner, umifoxolaner, esafoxolaner, tigolaner, and/or modoflaner, is administered in particular for the treatment of conjunctivitis, blepharitis, ocular rosacea, pterygium, chalazion, hordeolum, madarosis (to improve eyelash density, health, and appearance) or other indications including other ocular surface diseases such as meibomian gland dysfunction or dry eye disease. These can also be treated to improve visual acuity outcome prior to refractive or cataract surgery.

Some embodiments can include derivatives, analogues, and L- and D-isomers of isoxazoline parasiticide, including but not limited to enantiomers, compositions comprising racemic mixtures, and enantiomerically pure compositions.

In some embodiments, a dose of isoxazoline parasiticide can surprisingly be used that is lower than what has been shown to be clinically effective in veterinary medicine, which acts via systemic absorption via topical rinses or washes (e.g., at concentrations in the 1-10 nM, or 100 pM-1 nM range), or ranges including any two of the foregoing values. These lower effective concentrations may be, without limitation, due to direct absorption of drug by the mite body rather than ingestion of drug by the mite, with the abdomen of the mites being thinner (~0.5 um) and more likely to absorb drug than the mites' cephalothorax (~2 um). In some embodiments, direct absorption of drug by the mite body can be the mechanism responsible for at least about 50%, 60%, 70%, 80%, 90%, or more of the total uptake of the drug by the mite.

In some embodiments, daily and local treatment is administered rather than a large, long-acting systemic dose (as has been done in veterinary medicine once a month, every 8 weeks, every 12 weeks, every 16 weeks, or less frequently). However, long-acting systemic or local doses could be used in other embodiments.

In some embodiments, dosing could be, for example, about or at least about 1, 2, 3, 4, 5, 6, 7, 8, or more times daily, such as 1 to 2 times daily. In some embodiments, therapy could also be weekly, single dose or a limited-course of treatment. In some embodiments, a formulation can be preferentially used in the morning, at night, or only at night, to target exposure of mites during mating hours.

In some embodiments, formulations can be advantageous in part due to the slow elimination rate of molecules such as isoxazoline parasiticide, however, a small and local dose allows the repeated and frequent dosing, which may be advantageous to disrupt the Demodex life cycle through effects on more susceptible juvenile forms, without associated systemic risks and side-effects.

In some embodiments, an active molecule may preferentially be hydrophobic so it concentrates in regions with either sebum or meibum oils (e.g., eye lash follicles and/or meibomian glands). A formulation may be preferentially water-based to facilitate delivery to and absorption by the hydrophilic chitinous chitosan exterior of Demodex mites.

In some embodiments, a therapeutic agent can be delivered in the form of a drop, cream, ointment, eye wash, wipe, salve, or gel, or immediate or sustained release formulation. In some embodiments, a therapeutic agent can be delivered in the form of a punctal or canalicular plug or emulsion. In some cases, a form of an oily, gel-like, viscous ointment may also impede Demodex mite movement across the skin surface during mating.

In some embodiments, an isoxazoline parasiticide formulation may have preferential selectivity for the receptors of insects/mites/acari over vertebrate/mammalian/human receptors.

In some embodiments, an active agent is delivered in an oral formulation (e.g., tablet, capsule, solution, etc.), and a very small dose may be delivered to avoid meaningful systemic exposure or non-local dermal exposure (in contrast to veterinary teachings). However, in some embodiments, an active agent is delivered in a non-oral formulation, such as a topical formulation, e.g., a topical ophthalmic formulation.

In some embodiments, a dose of between, for example, 1 microgram/ml-1 mg/ml or 0.0001%-1% active agent (e.g., isoxazoline parasiticide) by weight, or between about 0.01% and 10% by weight, between about 0.05% and about 0.5% by weight, or about 0.01%, 0.015%, 0.02%, 0.025%, 0.03%, 0.04%, 0.05%, 0.06%, 0.07%, 0.08%, 0.09%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.00%, or ranges including any two of the aforementioned values, or 1 ng/ml-1 microgram/ml or 0.0000001-0.0001% active agent (e.g., isoxazoline parasiticide) by weight, or 1 mg/ml-100 mg/ml or 0.1-10% active agent (e.g., isoxazoline parasiticide) by weight, or ranges including any of the foregoing values. In some embodiments, the isoxazoline parasiticide is the only active agent.

In some embodiments, an ophthalmic formulation can be configured for delivery directly onto an ocular surface, including but not limited to the conjunctiva and/or cornea of the eye. In some embodiments, an ophthalmic formulation can be configured for delivery directly or indirectly onto any number of the anterior or posterior eyelids, eye lashes, or eyebrows. In some embodiments, an ophthalmic formulation is not directly delivered to any number of the conjunctiva, cornea, anterior or posterior eyelids, eye lashes, or eyebrows.

An eyedrop formulation may be designed to specifically and simultaneously treat blepharitis and Demodex in both the eyelash follicles and/or the meibomian glands, without limitation due to oily additives, emulsions, ointment or cream based formulations, delivery instrument such as lash brushes, or site of application. In some embodiments, a “Drop and Coat the Lashes” (DACTL) technique can be used. A patient can be instructed to close the eye(s) upon administration, thus causing the formulation to come into contact with the orifices of the meibomian glands on the margin(s) of the eyelid, and for the formulation to accumulate outside of the lid margin. The patient can then utilize their finger or an applicator to spread formulation which has accumulated outside of the lid margin onto the eye lashes and/or follicles of the eye lashes on the lower and/or upper eye lashes. Not to be limited by theory, as Demodex mites reside in both the eye lash hair follicles and in the meibomian glands, it can be advantageous for the eye drop formulation to be directly applied to one or both of these locations. Since these two targets in combination are unique to this disease, a therapeutic agent can be delivered to these locations simultaneously. The meibomian gland orifices are on the superior and inferior surfaces of the lower and upper eyelids, respectively. An eye drop placed directly onto the ocular surface thus allows for delivery of formulation directly to the upper and lower meibomian gland orifices. Optionally, a patient can also rub the eyelid margin to effect a further mechanical debridement of collarettes and mites residing in the eyelashes and meibomian glands, further enhancing the pharmacologic effects.

In some embodiments, a method of treating blepharitis and/or Demodex infestation does not involve or require lid scrubbing and/or wipes. Some wipes or lid scrub regimens including CLIRADEX (Bio-Tissue, Inc., Miami, FL) with includes tea tree oil can be very irritating to the eye, temporarily or otherwise, even when using terpinen-4-ol as the active ingredient alone. Such methods that do not involve lid scrubbing and/or wipes can advantageously be simpler, more expedient, and result in improved patient compliance. However, some embodiments can include wipes or lid scrub regimens.

In some embodiments, a method of treating blepharitis and/or Demodex infestation does not involve or require chafing with abrasive particles, powders, or crystals or otherwise performing microdermabrasion on the eye area tissue, such as the eyelids, eyelashes, or follicle areas. However, in some embodiments, any of the methods disclosed herein can include one or more of the following treatment modalities.

In some embodiments, a method of treating blepharitis and/or meibomian glad dysfunction that does not involve mechanical (e.g., vibratory), electromagnetic (e.g., RF, microwave, laser, or ultrasound) or heat based treatment of the eyelid, such as the LipiFlow Thermal Pulsation System (Johnson & Johnson Vision, Morrisville, NC), iLux (Tearfilm Innovations, Inc., Carlsbad, CA) or BlephEx (Scope Ophthalmics, West Sussex, UK) devices. However, in some embodiments, any of the methods disclosed herein can include one or more of the following treatment modalities.

Methods of treating blepharitis and/or Demodex infestation can include a formulation/treatment (or similar) delivered specifically by applying a drop in the eye, and then using a finger or instrument (e.g., lash brush) to coat the base of hair follicles in the upper and/or lower eyelids. In some embodiments, desirable features of a formulation can include any number of maximizing drug aqueous solubility to enhance bioavailability (in solution and suspensions), improve the residence time of the drug product in the eye using polymers/viscosity agents, and achieve acceptable visual acuity and comfort.

The viscosity of the formulation may be sufficiently high in some embodiments to cause coverage of formulation over meibomian gland orifices on upper and/or lower lid margins upon blinking or close of the eye.

In some cases, viscosity may be sufficiently high to slow evacuation of formulation through the puncta of the eye for at least 5 seconds, or 10 seconds or 20 seconds or 30 seconds or longer, to enhance contact time of formulation with meibomian gland orifices and to cause the formulation to spill over the lid margin to where it can be accessed for delivery to the eyelash follicles (e.g., by runoff, and/or by spreading of formulation using a finger and/or instrument).

Not to be limited by theory, in some patients with meibomian gland dysfunction (MGD), the quantity, quality and/or composition of the meibum changes. As such, some patients may have lipid deficiency in their meibum. Further, in meibomian gland dysfunction, the quality of expressed lipid varies in appearance from a clear fluid, to a viscous fluid containing particulate matter and densely opaque, toothpaste-like material. The meibomian orifices may exhibit elevations above surface level of the lid, which is referred to as plugging or pouting, and can be due to obstruction of the terminal ducts and extrusion of a meibum lipids of increased viscosity.

Lipid deficiency and/or increased viscosity of meibum can in some cases important pathogenic factors in MGD and be observed in cases of obstructive MGD. Therefore, it can be desirable in some cases to enhance lipogenesis and lipid secretion from the meibomian gland, to overcome lipid deficiency as well as reduce the viscosity of meibum oil composition which allows for dissolution of any obstruction of the meibomian gland.

In some embodiments, formulations and methods can be utilized to treat or prevent MGD, blepharitis, or other conditions as disclosed, for example, elsewhere herein, that include a therapeutically-effective amount of at least one or more of an isoxazoline parasiticide, as well as one, two, or more of a lipid-producing agent, keratolytic agent, and/or a mucolytic agent as disclosed, for example, in U.S. Pub. No. 2019/0125766 to Alster et al. and 2017/0087179 to Amselem et al., both of which are hereby incorporated by reference in their entireties.

Highly viscous meibum can be mixed with hyperkeratotic cell material, as seen in expressed pathologic human meibum prepared as smears or in impression cytology and in histopathology, as verified by molecular biology and immunohistochemistry. Increased viscosity has also been observed inside the obstructed glands of animal models. It can thus be desirable in some cases to soften and liquefy the obstructing lipids in order to open the duct and restore normal flow of excreted lipids.

Thiol-containing, —SeH (e.g., selenol) containing, and/or disulfide-containing drugs can increase the production of lipids in meibomian glands and/or increase the secretion of lipids from meibomian glands to the eyelid. This can be unexpectedly synergistically effective in preventing, treating and/or ameliorating certain adverse eyelid conditions, such as MGD, if combined with one or agents therapeutically effective against Demodex. Some embodiments could include a combination therapy of one or more of thiol-containing, —SeH containing, and/or disulfide-containing drugs, plus a therapeutically-effective amount of at least one or more of an isoxazoline parasiticide. The combination therapy could be present in a single combination formulation or a separate formulation delivered separately.

Some treatments for MGD include the use of mucolytic and/or keratolytic agents. One goal of mucolytic therapy is to facilitate physiological clearance by optimizing the viscoelasticity of mucus, while keratolytic therapy aims to soften keratin, a major component of the skin. This can be unexpectedly synergistically effective in preventing, treating and/or ameliorating certain adverse eyelid conditions, such as MGD, if combined with one or agents therapeutically effective against Demodex. Some embodiments could include a combination therapy of one or more of mucolytic and/or keratolytic agents, plus a therapeutically-effective amount of at least one or more of an isoxazoline parasiticide. The combination therapy could be present in a single combination formulation, or a separate formulation delivered separately.

In some embodiments, the keratolytic agent could include one or more of selenium sulfide, dithranol, benzoyl peroxide, urea, salicylic acid, boric acid, lactic acid, retinoic acid, and an alpha-hydroxy acid. However, in some embodiments, a therapy does not include any mucolytic and/or keratolytic agents.

Acetylcysteine, also known as N-acetylcysteine or N-acetyl-L-cysteine (NAC), can also be utilized as a mucolytic agent. This action of breaking disulfide bonds can be useful in thinning the abnormally thick mucus in, for example, cystic and pulmonary fibrosis patients. A formulation or method could include, for example, acetylcysteine plus a therapeutically-effective amount of at least one or more of an isoxazoline parasiticide. The combination therapy could be present in a single combination formulation, or a separate formulation delivered separately.

In some embodiments, disclosed is a method for increasing lipid secretion from a meibomian gland. This can be unexpectedly synergistically effective in preventing, treating and/or ameliorating certain adverse eyelid conditions, such as MGD, if combined with one or agents therapeutically effective against Demodex. In some embodiments, disclosed herein are formulations and methods including topically administering to the eyelid margin of the patient in need thereof an ophthalmic composition comprising an ophthalmically-acceptable carrier and a therapeutically-effective amount of at least one agent which increases lipogenesis in the meibomian gland or increases lipid secretion from the meibomian gland, wherein the agent comprises a sulfhydryl group or a disulfide, along with a therapeutically-effective amount of at least one or more of an isoxazoline parasiticide. The combination therapy could be present in a single combination formulation, or a separate formulation delivered separately.

In some embodiments, disclosed herein are formulations and methods for treating meibomian gland dysfunction (MGD), comprising topically administering to the eyelid margin of the patient in need thereof an ophthalmic composition comprising an ophthalmically-acceptable carrier and a therapeutically-effective amount of at least one or more of the following agents: captopril, zofenopril, tiopronin, penicillamine, glutathione, dithiothreitol, thiorphan, cysteamine, bucillamine, dimercaprol, 1,1-ethanedithiol, dimercaptosuccinic acid, furan-2-ylmethanethiol, omapatrilat, ovothiol A, pantetheine, rentiapril, thiosalicylic acid, tixocortol, mycothiol, coenzyme A, and coenzyme B, or combinations thereof. In some embodiments, the formulation can include a disulfide, as well as a therapeutically-effective amount of at least one or more of an isoxazoline parasiticide. The combination therapy could be present in a single combination formulation, or a separate formulation delivered separately.

In some embodiments, a therapeutic agent comprises a disulfide bond. In certain embodiments, the agent can be one or more of: disulfiram, psammaplin A, dixanthogen, pantethine, fursultiamine, octotiamine, sulbutiamine, prosultiamine, thiram, lipoic acid, lenthionine, ajoene, allicin, gemopatrilat, and sulfanegen. A formulation including one or more of the aforementioned agents can be utilized together with a therapeutically-effective amount of at least one or more of an isoxazoline parasiticide. The combination therapy could be present in a single combination formulation, or a separate formulation delivered separately.

In some embodiments, disclosed herein are methods for lowering the melting point of lipids secreted from a meibomian gland, comprising topically administering to the eyelid margin of the patient in need thereof an ophthalmic composition comprising an ophthalmically-acceptable carrier and a therapeutically-effective amount of at least one agent which increases lipogenesis in the meibomian gland or increases lipid secretion from the meibomian gland, wherein the agent comprises a sulfhydryl group, —SeH group, or a disulfide, as well as a therapeutically-effective amount of at least one or more of isoxazoline parasiticide. The combination therapy could be present in a single combination formulation, or a separate formulation delivered separately.

In some embodiments, disclosed herein are methods for reducing the viscosity of lipids secreted from a meibomian gland, comprising topically administering to the eyelid margin of the patient in need thereof an ophthalmic composition comprising an ophthalmically-acceptable carrier and a therapeutically-effective amount of at least one agent which increases lipogenesis in the meibomian gland or increases lipid secretion from the meibomian gland, wherein the agent comprises a sulfhydryl group, —SeH group, or a disulfide, as well as a therapeutically-effective amount of at least one or more of an isoxazoline parasiticide. The combination therapy could be present in a single combination formulation, or a separate formulation delivered separately.

In certain embodiments, a formulation can include one or more agents including a thiol group, or —SeH group. In certain embodiments, the agent is selected from the group consisting of captopril, zofenopril, tiopronin, penicillamine, L-cysteine, selenocysteine, glutathione, dithiothreitol, thiorphan, cysteamine, bucillamine, dimercaprol, 1,1-ethanedithiol, dimercaptosuccinic acid, furan-2-ylmethanethiol, omapatrilat, ovothiol A, pantetheine, rentiapril, thiosalicylic acid, tixocortol, mycothiol, coenzyme A, and coenzyme B. Any of the foregoing can be delivered in combination with a therapeutically-effective amount of at least one or more of an isoxazoline parasiticide. The combination therapy could be present in a single combination formulation, or a separate formulation delivered separately. In some embodiments, also disclosed herein is a method for reducing the viscosity of lipids in an eyelid margin, comprising topically administering to the eyelid margin of the patient in need thereof an ophthalmic composition comprising an ophthalmically-acceptable carrier and an effective amount of at least one lipid-derivative comprising a sulfhydryl group or a disulfide. Any of the foregoing can be delivered in combination with a therapeutically-effective amount of at least one or more of an isoxazoline parasiticide. The combination therapy could be present in a single combination formulation, or a separate formulation delivered separately.

One embodiment provides a method for enhancing lipogenesis and lipid secretion from a meibomian gland in a patient in need thereof by administering a topical composition comprising a lipid-derivative, wherein the composition comprises 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.5%, 5%, or 10% of the lipid-derivative, or ranges including any two of the foregoing values. In some embodiments, the composition is formulated as a suspension, emulsion, cream, lotion, gel, or ointment. In some embodiments, the composition is applied as a thin layer to clean skin initially once daily on alternate days and is then gradually increased up to twice daily as tolerance develops. In some embodiments, the composition is an ointment or paste. In some embodiments, the composition is started as a 0.1% ointment. After 7 days, the concentration may be increased to 0.25% and subsequently doubled, if necessary, at weekly intervals to a maximum strength of 2%. In some embodiments, a thin layer of ointment is applied once daily to the affected areas for 2-4 weeks. In some embodiments, the ointment is left in place for 10 to 20 minutes before the area is rinsed thoroughly. In some embodiments, the concentration of lipogenesis and lipid secretion enhancing thiol-containing or disulfide-containing drug or pharmaceutical agent is gradually increased to a maximum of 5% or more, and treatment is continued for as long as necessary.

In certain embodiments, the lipid-derivative is a derivative of a lipid selected from the group consisting of a fatty acid, a glycerolipid, a glycerophospholipid, a sphingolipid, a sterol lipid, a prenol lipid, a saccharolipid, a polyketide, and any combination thereof. Each possibility represents a separate embodiment of the invention. In certain embodiments, the lipid-derivative is a derivative of a lipid found naturally in the meibum.

In some embodiments, the lipid derivative is a lipid containing a —S—H or disulfide such as Thiophospholipid, Thiocholesterol, 12-Mercaptododecanoic acid or 23-(9-Mercaptononyl)-3,6,9,12,15,18,21-Heptaoxatricosanoic Acid.

Another embodiment provides the method for increasing lipid secretion from a meibomian gland wherein the lipid is a phospholipid and the phospholipid is selected from the group consisting of phosphatidylcholine (PC), phosphatidylethanolamine (PE), alkylacylphosphatidylcholine, sphingomyelin, dihydrosphingomyelin, dimethylphosphatidylethanolamine, diphosphatidylglycerol (cardiolipin), ethanolamine plasmalogen, lysoethanolamine plasmalogen, lysophosphatidylcholine, lysophosphatidylethanolamine, lysophosphatidylserine, phosphatidic acid, phosphatidylglycerol, phosphatidylinositol, and phosphatidylserine.

Another embodiment provides the method for increasing lipid secretion from a meibomian gland wherein the lipid is a fatty acid amide and the fatty acid amide is selected from the group consisting of oleamide, myristamide, palmitamide, stearamide, erucamide and ceramide.

Any of the features disclosed herein can be delivered in combination with a therapeutically-effective amount of at least one or more of an isoxazoline parasiticide. The combination therapy could be present in a single combination formulation, or a separate formulation delivered separately.

Formulation constituents can be chosen to enable dissolution of active agent into a solution, but with a low concentration by weight of organic solvents, e.g., ≤50%, 20%, 10%, 5%, 2%, or 1%, or less or more by weight organic. This may be achieved at least in part by using a surfactant and/or solubilizing agent such as, for example, polysorbate-80 or polysorbate-20. In some cases, low concentrations of polysorbate 80 may be preferentially used, since higher concentrations may lead to isoxazoline parasiticide hydrolysis (e.g., 0.001-0.1% polysorbate 80 by weight).

In some cases, this can also be achieved and solubility of isoxazoline parasiticide enhanced through organic solvents such as propylene glycol.

Solubility and viscosity may be also simultaneously enhanced by selection of an appropriate additive, thereby minimizing osmolarity, e.g., with polyvinyl alcohol, carboxymethylcellulose or the like.

Formulation constituents can be chosen to enable dissolution of active agent (e.g., isoxazoline parasiticide or other active agents) into a solution, and one that is stable from hydrolytic reactions for up to 1, 1.5, 2 years, or more to enable commercial shelf life e.g., with an optimal pH range of neutral to slightly alkaline (e.g., pH 7-10, 7-7.5), or slightly acidic (e.g., pH 5-7 in other embodiments).

The buffer concentration required to achieve the desired pH can be minimized in some cases, and thus retarding the hydrolysis rate (e.g., phosphate buffer concentration 0.01-0.1M). This may also be achieved with organic solvents and surfactants and/or solubilizing agents at concentration ranges described above.

Cationic surfactants and/or solubilizing agents, through creation of cationic micelles, can also be advantageous by retarding the hydrolysis rate.

Emulsions and emulsifiers may be mixed with water to shield isoxazoline parasiticide from water in an oil-in-water droplet, e.g., with a carbodiimide additive to prolong stability by forming more complex water-free micelles.

Water scavengers such as Stabaxol I® (bis-2,6-diisopropylphenylcarbodiimide) may be added to achieve long-term oil-based formulations to clean solvents of water.

The pharmaceutical compositions in some embodiments can comprise at least one compound selected from among the family of the isoxazoline parasiticide, wherein the pharmaceutical compositions are particularly useful for the treatment of ophthalmic symptoms, and wherein the symptoms are selected from a feeling or sensation of burning or of smarting of the eye, a feeling or sensation of a foreign body in the eye, a feeling or sensation of dryness of the eye, an increased sensitivity to light, blurred vision, telangiectasia of the eyelid margin, meibomitis, chalazia, conjunctival hyperemia and papillary conjunctivitis.

The term “treatment” can include treatment in humans and/or other animals.

The pharmaceutical compositions according to some embodiments of the invention can be useful for the treatment of the eyes topically, orally, parenterally or rectally.

The topical application is the most common method of administration of ophthalmic medicaments. The topical route makes possible the instillation into the eye of drops or the application in the eye of solutions, eyewashes, suspensions, salves, ointments, gels, sprays, foams, powders, lotions, viscoelastic solutions and/or the deploying of solid forms at the surface of the eye, impregnated pads, syndets or wipes.

Some formulations can also be provided in the form of suspensions of microspheres or nanospheres or of vesicles formed from lipid or polymer or of polymeric patches and of hydrogels making possible controlled release. These compositions for topical application can be provided in anhydrous form, in aqueous form or in the form of an emulsion.

The pharmaceutical compositions for topical application are preferably non-irritating and compatible with the tissues of the eye. The solutions can be sterile and can be free from all particles. The suspensions can be sterile and can include solid particles in a liquid vehicle appropriate for ocular instillation. The ointments can be semisolid and sterile preparations.

Orally, the pharmaceutical compositions can be provided in liquid, pasty or solid form, in the form of powders and more particularly in the form of tablets, including sugar-coated tablets, hard gelatin capsules, syrups, suspensions, solutions, powders, granules, emulsions, microspheres or nanospheres or vesicles formed from lipid or polymer making possible controlled release. Parenterally, the compositions can be provided in the form of solutions or suspensions for infusion or for injection. Rectally, the compositions can be provided in the form of suppositories. In some cases, the pharmaceutical compositions are topical ophthalmic compositions, and not oral or rectal compositions.

The compositions can in some embodiments comprise from 0.001% to 10% of at least one compound selected from the family of isoxazoline parasiticide, by weight with respect to the total weight of the composition. In some embodiments, the compositions according to the invention comprise from 0.01% to 5% of at least one compound selected from the family of the isoxazoline parasiticide, by weight with respect to the total weight of the composition.

In some embodiments, the compositions according to the invention are provided in the form of an eyewash or of eye drops. The term “eyewash” means a liquid formulation specifically appropriate for administration to the conjunctiva of the eye and the cornea. The eyewash can include a volume of the instilled drops of, e.g., approximately 25-50 microliters, or about 5, 10, 15, 20, 25, 30, 35, 40, 45, 50, 55, 60, 65, 70, or 75 microliters, or ranges including any two of the foregoing values. In some embodiments, the total volume of the formulation delivered in a treatment session (e.g., per day, twice a day, three times a day, or other time interval as disclosed elsewhere herein) is about or no more than about 200 microliters, 150 microliters, 100 microliters, 75 microliters, 50 microliters, 25 microliters, or more or less. In some embodiments, compositions are supplied as a kit, for example, an eyedrop and shampoo, and may be used along with sterilizing agents such as tea tree oil and derivatives, and hypochlorous acid, which have also been shown to have Demodex activity, or not include tea tree or other oils in some cases.

As indicated above, the compositions can in some embodiments meet specific conditions in order to be applied in the eye. Such conditions include, in particular, sterility, absence of irritation and compatibility with the tissues of the eye. The latter criterion is more difficult to obtain than for a composition applied to the skin; in particular, compounds such as ethanol or glycols, formulated in compositions to be applied to the skin, cannot in some cases be included in compositions for ocular use.

The topical compositions can make it possible to directly and specifically treat the symptoms of the pathology in the eye and eyelids by a local action; in particular, since only the eye is targeted, a better effectiveness can be expected.

In some embodiments, a formulation can be in a solution, suspension, cream, ointment, or other form.

A liquid composition, which is formulated for topical ophthalmic use, is formulated such that it can be administered topically to the eye. The comfort may be maximized as much as possible, although sometimes formulation considerations (e.g., drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use or contain a preservative to prevent contamination over multiple uses.

For ophthalmic application, solutions or medicaments are often prepared using a physiological saline solution as a major vehicle. These vehicles include but are not limited to polyvinyl alcohol, povidone, hydroxypropyl methyl cellulose, poloxamers, carboxymethyl cellulose, hydroxyethyl cellulose and purified water. Ophthalmic solutions may preferably be maintained at a comfortable pH with an appropriate buffer system. The formulations may also contain pharmaceutically acceptable preservatives, stabilizers, surfactants and/or solubilizing agents.

In some embodiments, a topical formulation does not include a dermal penetration enhancer, which could increase systemic absorption and be contrary to the intent of maintaining the formulation locally at or proximate the site of application in some cases. In some embodiments, a formulation does not include any dermal penetration enhancers, such as one or more of Laurocapram (Azone®) and laurocapram derivatives, such as 1-alkylazacycloheptan-2-ones, and oleic acid and its ester derivatives, such as methyl, ethyl, propyl, isopropyl, butyl, vinyl and glyceryl monooleate, and sorbitan esters such as sorbitan monolaurate and sorbitan monooleate, and other fatty acid esters such as isopropyl laurate, isopropyl myristate, isopropyl palmitate, diisopropyl adipate, propylene glycol monolaurate and propylene glycol monooleate, and long chain alkyl esters of 2-pyrrolidone, particularly the 1-lauryl, 1-hexyl and 1-(2-ethylhexyl) esters of 2-pyrollidene and those dermal penetration enhancers such as dodecyl (N,N-dimethylamino) acetate and dodecyl (N,N-dimethylamino) propionate and 2-n-nonyl-1-3-dioxolane. However, some embodiments of formulations can include one or more dermal penetration enhancers.

In some embodiments, a topical formulation can include one or more gelling agents. The gelling agent could be a copolymer, such as Pemulen™ TR1 and/or TR2 polymeric emulsifiers (Lubrizol Corp., Wickliffe, OH) which are high molecular weight, copolymers of acrylic acid and C10-C30 alkyl acrylate crosslinked with allyl pentaerythritol. They are fluffy, white powders and are primarily used to form stable oil-in-water emulsions. Pemulen polymers include both hydrophilic and hydrophobic portions within the molecule. The hydrophobic portion of the polymer adsorbs at the oil-water interface, and the hydrophilic portion swells in the water forming a gel network around the oil droplets to provide emulsion stability. Pemulen polymers can form stable oil-in-water emulsions without the need for any additional surfactants and/or solubilizing agents. Therefore, they can be advantageous for developing low irritancy lotions and creams, for example. Pemulen polymers provide viscosity building and high yield value to allow for suspension and stabilization of insoluble materials and particulates. In some embodiments, the gelling agent can be absent, or present in the formulation between about 0.001% and about 1%, between about 0.01% and about 0.10%, or about 0.001%, 0.005%, 0.01%, 0.02%, 0.03%, 0.04%, 0.05%, 0.06%, 0.07%, 0.08%, 0.09%, 0.10%, 0.15%, 0.20%, 0.25%, 0.30%, 0.35%, 0.40%, 0.45%, 0.50% w/w of the formulation, or ranges including any two of the foregoing values.

In some embodiments, an oil-based formulation such as, for example, a cream, ointment, or emulsion can include one or more of mineral oil, castor oil solubilizing agent, or petrolatum, such as between about 20% and about 80%, or between about 30% and about 70% w/w of the formulation. The formulation can also include a cyclodextrin as a carrier molecule to facilitate dissolution.

In some embodiments, a topical formulation can include one or more thickening agents, including a polysaccharide thickener/emulsant, such as hydroxypropylmethylcellulose (HPMC) and sodium CMC, polyvinyl alcohol, polyvinylpyrrolidone, dextran 70, or others. In some embodiments, the thickening agent can be present between about 0% and about 2%, between about 0.10% and about 1.00%, between about 0.25% and about 1.00%, or about 0.10%, 0.20%, 0.25%, 0.30%, 0.35%, 0.40%, 0.50%, 0.60%, 0.70%, 0.80%, 0.90%, 1.00% w/w of the formulation, or ranges including any two of the foregoing values, such as between about 0.1% and about 0.5%. In some embodiments, the formulation can have a viscosity of between about 50 cP and about 100 cP in order to increase the residence time of the formulation in the eye. In some embodiments, a formulation can include a viscosity of at or above 5 cP, 20 cP, 40 cP, 100 cP, 250 cP, 400 cP, 1000 cP or more, or ranges including any two of the foregoing values. In some cases, the formulation is configured to have a residence time in the eye of between about 90 seconds and about 10 minutes, or about or at least about 60 seconds, 90 seconds, 120 seconds, 180 seconds, 240 seconds, 300 seconds, 6 minutes, 7 minutes, 8 minutes, 9 minutes, 10 minutes, or ranges including any two of the foregoing values.

In some embodiments, a topical formulation can include one or more surfactants and/or solubilizing agents, including a non-ionic surfactant such as a polysorbate, such as Polysorbate 80, Polysorbate 65, Polysorbate 60, Polysorbate 40, or Polysorbate 20. In some embodiments, Polysorbate 80 has been found to unexpectedly result in increased solubility of an isoxazoline parasiticide over other polysorbates. Other surfactants, such as, for example, a fluorinated surfactant, can be substituted or used in addition to a non-ionic surfactant. In some embodiments, the solubilizing agent could be a sorbitan ester solubilizer, such as SPAN 20 (sorbitan monolaurate), SPAN 40 (sorbitan palmitic acid ester), SPAN 60 (sorbitan stearic acid ester), SPAN 80 (sorbitan oleic acid ester), SPAN 65 (sorbitan tristearic acid ester), and SPAN 85 (sorbitan trioleic acid ester). In some embodiments, the surfactants and/or solubilizing agents can be present between about 0% and about 5%, between about 0.10% and about 4%, between about 0.50% and about 4%, or about 0.50%, 0.75%, 1.00%, 1.25%, 1.50%, 1.75%, 2.00%, 2.25%, 2.50%, 2.75%, 3.00%, 3.25%, 3.50%, 3.75%, 4.00%, 4.25%, 4.50%, 4.75%, 5.00%, 5.25%, 5.50%, 5.75%, 6.00%, 6.25%, 6.50%, 6.75%, or 7.00% w/w of the formulation, or ranges including any two of the foregoing values. Polysorbate-80 may also be replaced with a castor oil solubilizing agent, up to 5%, 6%, or 7%, such as about 1%, 2%, 3%, 4%, or 5%, or between about 1% and 5%, between about 3% and about 7%, between about 5% and about 7%, or ranges including any two of the aforementioned values, to facilitate higher drug concentrations. Castor oil solubilizing agents have in some cases shown unexpectedly superior solubility of isoxazoline parasiticide compared with polysorbates.

In some embodiments, a formulation (including but not limited to an eye drop, cream, ointment, or other form as disclosed elsewhere herein) can include both a castor oil solubilizing agent and a polysaccharide thickener/emulsant such as HPMC or sodium CMC. In some cases, the combination can advantageously and unexpectedly form a long-lasting film layer.

In some embodiments, a formulation can include a castor oil solubilizing agent and not include a polysorbate solubilizing agent. Not to be limited by theory, in some embodiments a castor oil solubilizing agent has been found to provide unexpectedly beneficial solubility for relatively low solubility molecules, such as isoxazoline parasiticides, for example, including but not limited to fluralaner, sarolaner, lotilaner, afoxolaner, fluxametamide, isocycloseram, mivorilaner, umifoxolaner, esafoxolaner, tigolaner, or modoflaner, even beyond that of polysorbates. However, micelles of the castor oil solubilizing agent, at relatively high concentrations, such as, for example, between about 3% and about 7%, or between about 4% and about 5%, have been found to sequester and inactivate phosphate-based preservatives such as, for example, sodium phosphate dibasic heptahydrate or sodium phosphate monobasic monohydrate. As such, some embodiments do not include a phosphate buffer/preservative. Tromethamine (TRIS) in combination with benzalkonium chloride, for example, was surprisingly not found to inactivate a preservative and thus can be advantageous to use with a castor oil solubilizing agent in some cases.

In some embodiments, a topical formulation can include one or more tonicity agents, such as, for example, glycerin or other polyols, dextrose, mannitol, potassium chloride, and/or sodium chloride. Among the polyols, the 5-carbon xylitol, 4-carbon erythritol, and 3-carbon glycerol/glycerin can be preferred for ophthalmic use. The 6-carbon forms (mannitol, sorbitol, and related deoxy compounds) may be useful in combination with the smaller molecules. In one embodiment, combinations of polyols with 3 to 6 carbons, and 1 and 2 carbon deoxy derivatives including, without limitation, isomers, stereo-isomers and the like, as appropriate, may be utilized. Not to be limited by theory, polyols can be advantageous when used in combination with, for example, a castor oil solubilizing agent to disrupt the castor oil micelles and prevent inactivation of preservative agents such as BAK or LAK, of which sterility can be important for, e.g., multi-dose vial formulations. In some embodiments, uncharged or zwitterionic amino acids are useful as organic compatible solute components in accordance with the present invention. In some embodiments, carnitine components, for example, carnitine itself, isomers/stereo-isomers thereof, salts thereof, derivatives thereof and the like and mixtures thereof, can be useful compatible solute components for use in the present ophthalmic compositions. Carnitine is well-established as necessary for various parts of fatty acid metabolism, so it has a significant role in the metabolism of liver and muscle cells. Carnitine may serve as an energy source for many types of cells, including ocular cells. Carnitine components may have unique properties in multiple roles, for example, as osmoprotectants, in fatty acid metabolism, as an antioxidant, in promoting wound healing, as a protein chaperone, and in neuroprotection. In some embodiments, the tonicity agent(s) can be present between about 0% and about 5%, between about 0.10% and about 4%, between about 0.50% and about 4%, or about 0.50%, 0.75%, 1.00%, 1.25%, 1.50%, 1.75%, 2.00%, 2.25%, 2.50%, 2.75%, 3.00%, 3.25%, 3.50%, 3.75%, 4.00%, 4.25%, 4.50%, 4.75%, 5.00% w/w of the formulation, or ranges including any two of the foregoing values. In some embodiments, formulations can be stored in single-dose or multi-dose dispensers, including, for example, ophthalmic multi-dose bottles including formulations that do not include any preservatives, e.g., preservative-free bottles. Some embodiments utilize multi-dose preservative-free squeeze dispensing containers such as, for example, bottles, including but not limited to bottles including one-way valve dispensers.

In some embodiments, a topical formulation can include one, two, or more buffering agents. Buffering agents can include, for example, acetate buffers, citrate buffers, phosphate buffers and borate buffers. Acids or bases may be used to adjust the pH of these formulations as needed. The buffering agent could be one or more of sodium bicarbonate buffer, calcium bicarbonate buffer, tris(hydroxymethyl)aminomethane (Tris or THAM), MOPS (3-(N-morpholino) propanesulfonic acid) buffer, HEPES (N-(2-hydroxyethyl) piperazine-N′-(2-ethanesulfonic acid)) buffer, ACES (2-[(2-amino-2-oxoethyl)amino]ethanesulfonic acid) buffer, ADA (N-(2-acetamido)-2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid) buffer, Bicine (N,N-bis(2-hydroxyethylglycine)) buffer, Bis-Tris(bis-(2-hydroxyethyl)imino-tris(hydroxymethyl) methane) buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino) ethanesulfonic acid) buffer, DIPSO (3-[N,N-bis(2-hydroxyethyl)amino]-2-hydroxy-propanesulfonic acid) buffer, HEPPS (N-(2-hydroxyethylpiperazine)-N′-(3-propanesulfonic acid)) buffer, HEPPSO (N-(2-hydroxyethyl) piperazine-N′-(2-hydroxypropanesulfonic acid)) buffer, MES (2-(N-morpholino) ethanesulfonic acid) buffer, triethanolamine buffer, imidazole buffer, glycine buffer, ethanolamine buffer, phosphate buffer, MOPSO (3-(N-morpholino)-2-hydroxypropanesulfonic acid) buffer, PIPES (piperazine-N,N′-bis(2-ethanesulfonic acid)) buffer, POPSO (piperazine-N,N′-bis(2-hydroxypropanesulfonic acid)) buffer; TAPS (N-tris(hydroxymethyl)methyl-3-aminopropanesulfonic acid) buffer, TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine) buffer, 2-amino-2-methyl-1,3-propanediol buffer, and 2-amino-2-methyl-1-propanol buffer, as well as combinations thereof. In some embodiments, the buffering agent is Tris and/or disodium hydrogen phosphate (Na₂HPO₄) and sodium dihydrogen phosphate heptahydrate (NaH₂PO₄7H₂O). In some embodiments, the buffering agents can be present between about 0% and about 2%, between about 0.01% and about 1%, between about 0.01% and about 0.75%, or about 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.00% w/w of the formulation, or ranges including any two of the foregoing values. The buffering agents can be selected in a therapeutically effective amount such that the pH of the pharmaceutical composition can be, for example, between about 7.35 and about 7.65, between about 7.45 and 7.55, or about 7.30, 7.35, 7.40, 7.45, 7.50, 7.55, 7.60, or ranges including any two of the foregoing values.

In some embodiments, a topical formulation can include one or more preservative agents, including but not limited to lauralkonium chloride and benzalkonium chloride. Other preservatives can include, for example, PHMB, chlorobutanol, thimerosal, phenylmercuric, acetate and phenylmercuric nitrate. Still other preservatives can include, for example, a sorbate. The sorbate could include, for example, one or more of potassium sorbate, sodium sorbate, and calcium sorbate. Sorbates are distinct from polysorbates. In some embodiments, a formulation includes a sorbate, but does not include a polysorbate. In some embodiment, the sorbate could be the only preservative agent in the formulation. In some embodiments, a formulation does not include lauralkonium chloride and/or benzalkonium chloride. In some embodiments, the preservative agent can be present in the topical formulation between about 0.001% and about 0.5%, between about 0.001% and about 0.3%, between about 0.20% and about 0.50%, between about 0.001% and about 0.1%, between about 0.001% and about 0.01%, or about 0.001%, 0.002%, 0.003%, 0.004%, 0.005%, 0.006%, 0.007%, 0.008%, 0.009%, 0.010%, 0.05%, 0.10%, 0.15%, 0.20%, 0.25%, 0.30%, 0.35%, 0.40%, 0.45%, 0.50% w/w of the formulation, or ranges including any two of the foregoing values. In some embodiments, a formulation can be preservative-free, such as, for example, a single unit dose formulation, e.g., a single unit dose eye formulation. In some embodiments, a single unit dose can include a volume of between about 20 μL to about 300 μL, such as about 20 μL, 25 μL, 30 μL, 35 μL, 40 μL, 45 μL, 50 μL, 55 μL, 60 μL, 65 μL, 70 μL, 75 μL, 100 μL, 150 μL, 200 μL, 250 μL, 300 μL, or ranges including any two of the foregoing values.

The pharmaceutical compositions according to some embodiments of the invention can additionally comprise inert additives or combinations of these additives, such as: wetting agents, emollients; agents for improving flavor; preservatives; stabilizing agents; agents for regulating moisture; pH-regulating agents; buffers; agents for modifying osmotic pressure; emulsifying agents; agents for increasing viscosity; and antioxidants. Ophthalmically acceptable antioxidants include but are not limited to sodium metabisulfite, sodium thiosulfate, acetylcysteine, butylated hydroxyanisole and butylated hydroxytoluene. Other excipient components, which may be included in the ophthalmic preparations, are chelating agents. A useful chelating agent is ethylenediaminetetraacetic acid or salts or derivatives thereof, such as, for example, edetate or disodium (EDTA), although other chelating agents may also be used in place or in conjunction with it. In some embodiments, the EDTA is present at between about 0.001% and about 0.10% of the formulation, between about 0.01% and about 0.05% of the formulation, about 0.001%, 0.005%, 0.01%, 0.02%, 0.03%, 0.04%, 0.05%, or more or less, or ranges including any two of the foregoing values.

In some embodiments, a pharmaceutical composition can include a tocopherol. In some cases, a tocopherol can be effective in preventing degradation of an isoxazoline in water. In some cases, the vitamin E is a tocopherol; in a further embodiment the tocopherol is an alpha- or a gamma-tocopherol; more preferred is an alpha-tocopherol. In some embodiments, a pharmaceutical composition does not include a tocopherol.

In some embodiments, a pharmaceutical formulation does not include any essential oils, such as tea tree oil, alpha-Terpineol, Cardinene, d-Carvone, 1-Carvone, gamma-Terpinene, alpha-Terpinene, 1,8-Cineole, alpha-Terpineol, para-Cimene, alpha-Pinene, Limonene, alpha-Thugene, Eucalyptol, (+)-Ledene, Cuminic Aldehyde, terpinen-4-ol, Sabinene or Myrcene. Tea tree oil and derivatives can be quite irritating to the eye, resulting in potentially lower patient compliance. However, some embodiments can include one or more of the foregoing essential oils.

In some embodiments, a pharmaceutical formulation can also include neem oil and/or coconut oil. In some embodiments, a pharmaceutical formulation does not include neem oil and/or coconut oil.

In some embodiments, a pharmaceutical formulation can also include povidone-iodine (PVP-I) and/or dimethyl sulfoxide (DMSO). In some embodiments, a pharmaceutical formulation does not include PVP-I and/or DMSO.

In some embodiments, optional compound or compounds can be added to these compositions such that the advantageous properties intrinsically associated with some embodiments of the present invention are not, or not substantially, detrimentally affected by the envisaged addition, for example, tetracyclines or omega-3 fatty acids, which may have favorable effects in blepharitis.

In some embodiments, an isoxazoline parasiticide is administered orally to a patient with blepharitis, rosacea, or other conditions as disclosed elsewhere herein. Because one target organism, Demodex folliculorum (and/or Demodex brevis), is an ectoparasite in the mite family, an effective treatment in some cases is therapeutically eradicating the entire life cycle of such a microscopic insect, including egg, larval, and adult stages. For this reason, some embodiments treat blepharitis and/or rosacea patients with at least two doses timed so that between about three and about seven days separate the doses. Such spacing allows time for Demodex eggs to hatch into immature mites that are killed before they can mature into egg-producing adults. In some embodiments, 1, 2, 3, 4, or more doses at three- to seven-day intervals could be employed. After an isoxazoline parasiticide or other active agent as disclosed herein carries out its miticidal activity on skin Demodex folliculorum organisms (and/or Demodex brevis organisms), inflammatory responses to them begin to diminish but remnants of the dead mites still elicit some flushing and lesion formation until the cleanup processes of the body remove them, a process requiring six to eight weeks in some cases. During this initial phase of administration, other medications such as an antibiotic, including but not limited to oral tetracycline and topical metronidazole, and/or anti-inflammatory agents such as NSAIDs and/or steroids can be employed to suppress early flareups and to give early clinical response. However, in some embodiments, a formulation or method does not involve a tetracycline or other antibiotic, steroid, and/or metronidazole. After prolonged intervals of freedom from symptoms, should classic signs begin to reappear, treatment can be repeated.

In some embodiments, a formulation or method can include an anti-parasitic agent in combination with a steroid to synergistically reduce inflammatory symptoms. The steroid could be, for example, one or more of clobetasol, betamethasone, halobetasol, diflorasone, fluocinonide, halcinonide, amcinonide, desoximetasone, triamcinolone, mometasone, fluticasone, hydrocortisone, flurandrenolide, desonide, prednisone, prednisolone, dexamethasone, or alclometasone. The steroid could be delivered separately, or in the same formulation as the anti-parasitic agent. The steroid could have a concentration of about 0.001%, 0.005%, 0.01%, 0.025%, 0.05%, 0.1%, 0.2%, 0.25%, 0.5%, 0.75%, 1%, 1.5%, 2%, 2.5%, 3%, or more or less, or ranges including any two of the foregoing values. In some embodiments, a formulation does not include any steroids.

In some embodiments, a formulation can include an NSAID to reduce inflammatory symptoms. The NSAID could be, for example, one or more of pyrazolone/pyrazolidine NSAIDs, including aminophenazone ampyrone, azapropazone, clofezone, difenamizole, famprofazone, feprazone, kebuzone, metamizole, mofebutazone, morazone, nifenazone, oxyphenbutazone, phenazone, phenylbutazone, propyphenazone, sulfinpyrazone and suxibuzone; salicylate NSAIDs, including aspirin (acetylsalicylic acid), aloxiprin, benorylate, carbasalate calcium, diflunisal, dipyrocetyl, ethenzamide, guacetisal, magnesium salicylate, methyl salicylate, salsalate, salicin, salicylamide, salicylic acid (salicylate) and sodium salicylate; acetic acid derivative and related substance NSAIDs, including without limitation aceclofenac, acemetacin, alclofenac, amfenac, bendazac, bromfenac, bumadizone, bufexamac, diclofenac, difenpiramide, etodolac, felbinac, fenclozic acid, fentiazac, indomethacin, indomethacin farnesil, isoxepac, ketorolac, lonazolac, oxametacin, prodolic acid, proglumetacin, sulindac, tiopinac, tolmetin and zomepirac; oxicam NSAIDs, including ampiroxicam, droxicam, isoxicam, lornoxicam, meloxicam, piroxicam and tenoxicam; propionic acid derivative NSAIDs, including alminoprofen, benoxaprofen, carprofen, dexibuprofen, dexketoprofen, fenbufen, fenoprofen, flunoxaprofen, flurbiprofen, ibuprofen, ibuproxam, indoprofen, ketoprofen, loxoprofen, miroprofen, naproxen, oxaprozin, pirprofen, suprofen, tarenflurbil, tepoxalin, tiaprofenic acid and vedaprofen; N-arylanthranilic acid NSAIDs, including azapropazone, clonixin, ctofenamate, flufenamic acid, flunixin, meclofenamic acid, mefenamic acid, morniflumate, niflumic acid, tolfenamic acid and flutiazin; and/or coxib NSAIDs, including apricoxib, celecoxib, cimicoxib, deracoxib, etoricoxib, firocoxib, lumiracoxib, mavacoxib, parecoxib, robenacoxib, rofecoxib and valdecoxib. The NSAID could be delivered separately, or in the same formulation as the anti-parasitic agent. In some embodiments, a formulation does not include any NSAIDs.

In some embodiments, a formulation can include one, two, or more antibiotics, e.g., antibacterial agents. The antibiotics could include penicillin, including amoxicillin, ampicillin, bacampicillin, carbenicillin indanyl, mezlocillin, piperacillin, ticarcillin; penicillin and beta lactamase inhibitors, including amoxicillin-clavulanic acid, ampicillin-sulbactam, benzylpenicillin, cloxacillin, dicloxacillin, methicillin, oxacillin, penicillin G, penicillin VK, piperacillin+tazobactam, ticarcillin+clavulanic acid, nafcillin; first, second, third, or fourth generation cephalosporins; macrolides and lincosamides, including azithromycin, clarithromycin, clindamycin, dirithromycin, erythromycin, lincomycin, troleandomycin; quinolones and fluoroquinolones, including cinoxacin, ciprofloxacin, enoxacin, gatifloxacin, grepafloxacin, levofloxacin, lomefloxacin, moxifloxacin, nalidixic acid, norfloxacin, ofloxacin, sparfloxacin, trovafloxacin, oxolinic acid, gemifloxacin, pefloxacin; carbapenems, including imipenem, ertapenem, doripenem, and meropenem; monobactams, including aztreonam; aminoglycosides, including amikacin, gentamicin, kanamycin, neomycin, netilmicin, streptomycin, tobramycin, paromomycin; glycopeptides, including teicoplanin, vancomycin; tetracyclines, including demeclocycline, doxycycline, methacycline, minocycline, oxytetracycline, tetracycline, chlortetracycline; sulfonamides, including mafenide, silver sulfadiazine, sulfacetamide, sulfadiazine, sulfamethoxazole, sulfasalazine, sulfisoxazole, trimethoprim-sulfamethoxazole, sulfamethizole; rifampin, including rifabutin, rifampin, rifapentine; oxazolidinones, including linezolid; streptogramins, including quinupristin+dalfopristin; and others, including bacitracin, chloramphenicol, colistimethate, fosfomycin, isoniazid, methenamine, metronidazole, mupirocin, nitrofurantoin, nitrofurazone, novobiocin, polymyxin b, spectinomycin, trimethoprim, colistin, cycloserine, capreomycin, ethionamide, pyrazinamide, para-aminosalicylic acid, erythromycin ethylsuccinate+sulfisoxazole, and combinations thereof. In some embodiments, an antibiotic is therapeutically effective to treat Staphylococcus aureus (including MRSA), and/or Streptococcus, including S. pyogenes. In some embodiments, an antibiotic is therapeutically effective to treat Bacillus, including B. cereus and B. oleronius. The antibiotic could be delivered separately, or in the same formulation as the anti-parasitic agent. In some embodiments, a formulation does not include any antibiotics, e.g., antibacterial agents.

In some embodiments, a formulation can include a vasoconstrictor to reduce, for example, eye redness. The vasoconstrictor could include, for example, epinephrine, phenylephrine, norepinephrine, or methoxamine, or a combination thereof. The vasoconstrictor could be delivered separately, or in the same formulation as the anti-parasitic agent. In some embodiments, a formulation does not include a vasoconstrictor.

In an alternative embodiment, isoxazoline parasiticide can be formulated into a cosmetically acceptable topical lotion, cream, or gel and applied to skin, eyelids, eyelashes, meibomian glands, or other anatomical locations as noted elsewhere herein. In some cases, such a route of treatment can require once- or twice-daily applications for as long as four weeks to achieve sufficient follicle penetration and effective miticidal activity. A topical formulation that could achieve this effect could contain, for example, about 0.01-5% active ingredient in some cases and could be enhanced in penetration if the active agent were encapsulated inside microliposomes. Such a topical treatment would likely need to be repeated more frequently than the preferred oral embodiment, but a disease-free interval should be achieved by each course of therapy.

In some embodiments, an isoxazoline parasiticide can be incorporated into a gel formulation. The gel formulation could include a gelling agent. The gelling agent can be a carbomer gelling agent or a cellulosic gelling agent. One gelling agent can be hydroxyethyl cellulose (Natrosol, Hercules Inc., Wilmington, DE). Examples of other suitable gelling agents include carboxyvinyl polymers, such as Carbopol® 934, 940, or 941 (Noveon, Inc., Akron, OH). The polyacrylic acid gelling agent can be a carbomer gelling agent, such as, for example, Carbomer 940, 974P, or TR1. The carbomer gelling agent can be present in, for example, about 0.2-1 wt. %, such as, for example, about 0.25 wt. % or about 0.5 wt. %. In some embodiments, polyacrylic acid gel compositions include about 2.5-3.5 wt. % of benzyl alcohol. In some embodiments, the composition includes about 3 wt. % of benzyl alcohol. The viscosity of the carbomer composition can be, e.g., about 500 cps to about 32,000 cps, about 500 cps to about 15,000 cps, about 10,000 cps to about 15,000 cps, or about 26,000 cps to about 32,000 cps, depending on the amount of carbomer used in the formulation. The cellulosic gelling agent can be, for example, a hydroxyalkyl cellulose gelling agent, such as, for example, hydroxyethyl cellulose (HEC) or hydroxymethyl cellulose (HMC). The cellulosic gelling agent can be present in, for example, about 1-3 wt. %, such as, for example, about 1 wt. %, about 1.25 wt. %, about 1.5 wt. %, or about 2 wt. %. In some embodiments, the cellulosic gel compositions include about 2-3 wt. % of benzyl alcohol. In certain specific embodiments, the composition includes about 2.5 wt. % of benzyl alcohol.

In some embodiments, an isoxazoline parasiticide can be incorporated within an ocular insert. An ocular insert can be a sterile, thin, multilayered, drug-impregnated, solid or semisolid consistency device placed into the cul-de-sac or conjunctival sac, whose size and shape are especially designed for ophthalmic application. They can include a polymeric support that includes one, two, or more therapeutic agents such as an isoxazoline parasiticide or others as disclosed elsewhere herein. The therapeutic agent can later be incorporated as dispersion or a solution in the polymeric support. They can offer several advantages as increased ocular residence and sustained release of medication into the eye. The insert can include a body portion sized to position within a lacrimal canaliculus of the eyelid. The inserts are classified according to their solubility as insoluble, soluble, bioerodible, or non-erodible inserts. In some embodiments, an isoxazoline parasiticide can be incorporated within a punctal plug placed within the puncta or canaliculus, or a contact lens, or other device.

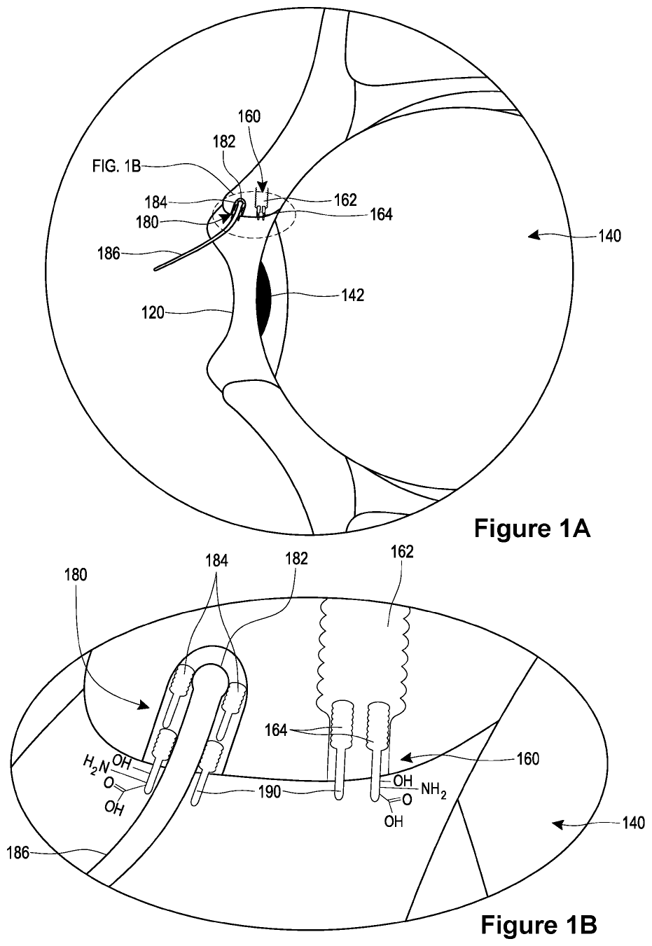

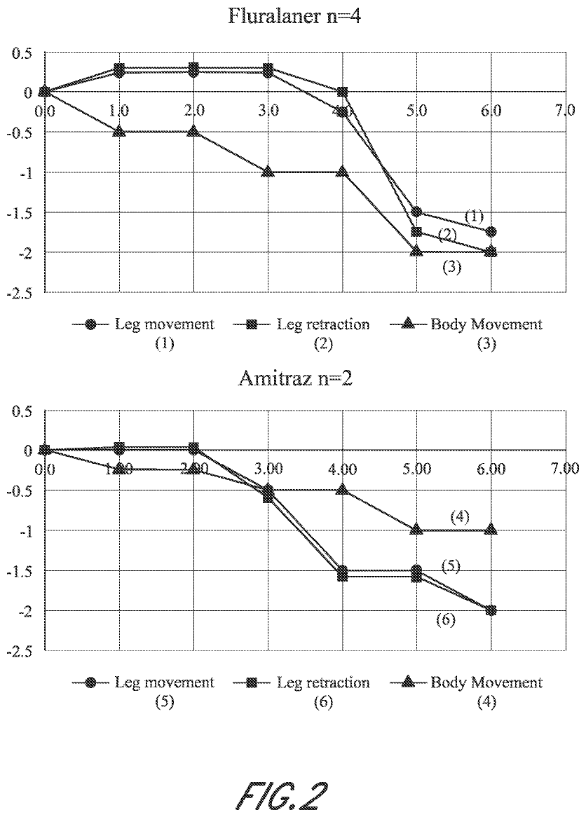

In some embodiments, the pharmaceutical formulation including any disclosed herein can be configured to advantageously allow for eradication of the mite proximate an eyelash via preferential absorption of the pharmaceutical formulation through the exoskeleton (e.g., abdomen or opisthosoma) of the mite rather than ingestion of the pharmaceutical formulation by the mite (e.g., by ingesting skin cells, sebum, and other elements that could include an amount of active agent via systemic absorption). Not to be limited by theory, Demodex mites have a hydrophobic chitin outer surface, and a relatively thin exoskeleton in the abdomen/opisthosoma area (about 0.5 μm, vs. about 2.0 μm for the cephalothorax portion), which surprisingly has allowed for more rapid absorption of the formulation through the abdomen, instead of primarily via ingestion as in previous veterinary formulations of isoxazoline parasiticide. FIGS. 1A-B schematically illustrate application of a formulation 120 onto an eye 140 with an iris/pupil 142. An eyelid around the eye 140 may include a hair follicle 180 for an eyelash 186 and the follicle 180 may include sebum oil 182. The eyelid may include a meibomian gland 160 with meibum oil 162. As shown in FIGS. 1A-B, a “face down” orientation of the mites (e.g., Demodex folliculorum 164, Demodex brevis 184) with respect to the hair follicle 180 or a Meibomian gland 160 (with the mite body pointing to the opening of the follicle 180 or the gland 160) may facilitate the preferential abdominal absorption of the formulation 120 through abdomen/opisthosoma area 190. In ex vivo studies, it has been surprisingly observed that following delivery of certain pharmaceutical formulations as disclosed herein that the abdomen and tail portion of a Demodex mite stops moving more quickly than the cephalothorax as in FIG. 2, indicating Demodex mites are especially susceptible to topical ophthalmic formulations as disclosed herein.

In some embodiments, compositions and methods as disclosed herein can include any number of the following agents, in topical or other forms, which can be made into formulations having parameters including any features including but not limited to concentrations, excipients, and other features or absence of other features as disclosed elsewhere herein: albendazole, cambendazole, fenbendazole, flubendazole, mebendazole, oxfendazole, parbendazole, tiabendazole, triclabendazole, amitraz, demiditraz, clorsulon, closantel, oxyclozanide, rafoxanide, cyphenothrin, flumethrin, permethrin, promazine, derquantel, diamphenetide, dicyclanil, dinotefuran, imidacloprid, nitenpyram, thiamethoxam, abamectin, doramectin, emamectin, eprinomectin, ivermectin, moxidectin, selamectin, milbemycin oxime, emodepside, epsiprantel, fipronil, fluazuron, fluhexafon, indoxacarb, levamisole, lufenuron, metaflumizone, methoprene, monepantel, morantel, niclosamide, nitroscanate, nitroxinil, novaluron, oxantel, praziquantel, pyrantel, pyriprole, pvriproxyfen, sisapronil, spinosyns and spinosoids (e.g., spinosyn A, B, C, D, E, F, G, H, J, K, L, M, N, O, P, Q, R, S, T, U, V, W, Y, and/or spinosad), spinetoram, lindane, picrotoxin, dieldrin, alpha-endosulfan, and/or triflumezopyrim. In some embodiments, compositions and methods can include a meta-diamide (e.g., broflanilide, tetraniliprole, or cyclaniliprole), a cyclodiene, and/or a phosphodiestaerase-4 inhibitor (including PDE4A, PDE4B, PDE4C, and/or PDE4D inhibitors, including but not limited to apremilast, cilomilast, crisaborole, diazepam, ibudilast, luteolin, mesembrenone, piclamilast, roflumilast, or rolipram) and/or a macrocyclic lactone (including avermectins and milbemycin). In some embodiments, formulations and methods can include an immunomodulatory agent (e.g., an immunostimulatory or immunosuppressive agent). An immunosuppressive agent can include, for example, a steroidal and/or non-steroidal anti-inflammatory agent. The non-steroidal anti-inflammatory agent can include, for example, a calcineurin inhibitor (e.g., tacrolimus, sirolimus, or pimecrolimus), cyclosporine, diclofenac, ibuprofen, ketoprofen, indomethacin, piroxicam, and the like. Derivatives, analogues, and L- and D-isomers thereof, including but not limited to enantiomers, compositions comprising racemic mixtures, and enantiomerically pure compositions of any of the foregoing in this paragraph can also be utilized. In some embodiments, a formulation does not include any number of, or all of the agents listed in this paragraph.

In some embodiments, a dermatologic and/or ophthalmologic formulation can include an active therapeutic agent of a formamidine parasiticide in addition to an isoxazoline parasiticide as disclosed above. A formamidine parasiticide can be, for example, amitraz, which can function as an octopamine receptor modulator. N-(2,4-Dimethylphenyl)-N-methyformamidine (DPMF), a metabolite of amitraz, is thought to be an active agent that exerts acaricidal and insecticidal effects by acting as an agonist on octopamine receptors, and can be another active therapeutic agent, alone or in addition. 2,4-dimethylanaline is a hydrolysis metabolite of DPMF and can also be an active therapeutic agent in other embodiments. Derivatives, analogues, and L- and D-isomers thereof, including but not limited to enantiomers, compositions comprising racemic mixtures, and enantiomerically pure compositions can also be utilized. In some embodiments, a dermatologic and/or ophthalmologic formulation can include an active therapeutic agent of a phenylpyrazole parasiticide in addition to an isoxazoline parasiticide as disclosed above. The chemical structures of these insecticides are characterized by a central pyrazole ring with a phenyl group attached to one of the nitrogen atoms of the pyrazole. Some non-limiting examples of phenyl pyrazole parasiticides include, for example, acetoprole, ethiprole, fipronil, flufiprole, pyraclofos, pyrafluprole, pyriprole, pyrolan, and vaniliprole.

In some embodiments, a formulation can include an organophosphate, which can have anti-Demodex activity, in addition to an isoxazoline parasiticide. The organophosphate could include one or more of, for example, acephate, azamethiphos, azinphos ethyl, azinphos methyl, bromophos, bromophos ethyl, cadusafos, carbophenothion, chlormephos, chlorphoxim, chlorpyrifos, chlorpyrifos-methyl, chlorthiophos, chlorvinphos, coumaphos, crotoxyphos, crufomate, cyanofenphos, cyanophos, demephron-O, demephron-S, demeton-O, demeton-S, demeton-S-methyl, demeton-S-methylsulphon, dialifos, diazinon, dichlofenthion, dichlorvos, dicrotophos, dimefox, dimethoate, dioxabenzofos, dioxathion, disulfoton, ditalimfos, edifenphos, EPBP, EPN, ESP, ethion, ethoprophos, etrimfos, famphur, fenamiphos, fenchlorphos, fenitrothion, fensulfothion, fenthion, fenofos, formothion, fosmethilan, heptenophos, isazofos, isofenphos, isothioate, isoxathion, iodofenphos, leptophos, metrifonate, malathion, menazon, mephosfolan, methacrifos, methamidophos, methidathion, mevinphos, monocrotophos, naled, omethoate, oxydemeton-methyl, parathion, parathion-methyl, phenthoate, phorate, phosalone, phosmet, phosphamidon, phosphamidon amide, phospholan, phoxim, pirimiphos-ethyl, pirimiphos-methyl, profenofos, propaphos, propetamphos, prothiofos, prothoate, pyraclofos, pyridaphenthion, quinalphos, schradan, sulfotep, sulprofos, temephos, TEPP, terbufos, tetrachlorvinphos, thiometon, thionazin, triazophos, trichlorfon, vamidothion, a prodrug of these and a pharmaceutically acceptable salt or ester of these. In some embodiments, the organophosphate can be dichlorvos or a prodrug or pharmaceutically acceptable salt or ester thereof. In some embodiments, the organophosphate can be metrifonate or a prodrug or pharmaceutically acceptable salt or ester thereof. In some embodiments, a formulation or method does not include any organophosphates.

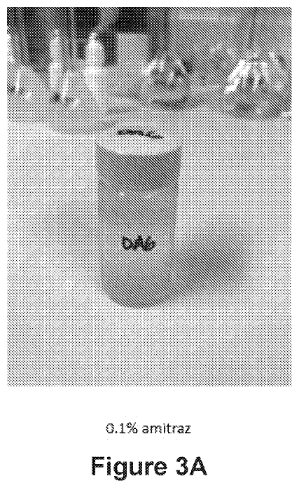

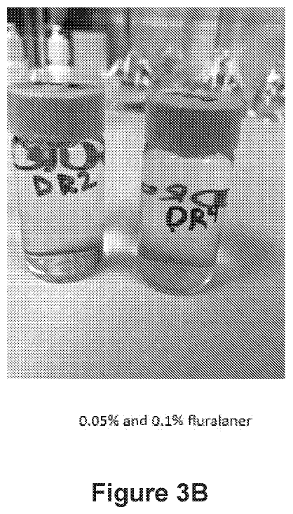

To further illustrate some embodiments and advantages thereof, Table 1 below lists several non-limiting specific examples of topical isoxazoline parasiticide formulations for illustrative purposes only. All ingredients are listed as % w/w or grams/100 grams of preparation. FIGS. 3A-3B shows examples of formulations with amitraz and fluralaner.

TABLE 1

| Ingredient | Solution 1 | Solution 2 | Suspension 1 |
|---|---|---|---|
| Isoxazoline parasiticide (e.g., | 0.0100 | 0.0250 | 0.500 |
| mivorilaner, umifoxolaner, | | | |
| esafoxolaner, tigolaner, or | | | |
| modoflaner) | | | |
| Pemulen TR1 | 0 | 0 | 0.050 |
| HPMC | 0.50 | 0.50 | 0 |
| Polysorbate 80 | 2.0 | 2.0 | 2.0 |
| Glycerin | 2.5 | 2.5 | 2.5 |
| TRIS | 0 | 0 | 0.050 |
| NaH₂PO₄7H₂O | 0.44 | 0.44 | 0 |
| Na₂HPO₄ | 0.045 | 0.045 | 0 |
| pH | 7.5 | 7.5 | 7.5 |
| Lauralkonium Chloride | 0.0050 | 0.0050 | 0.0050 |
| Water | q.s. ad 100% | q.s. ad 100% | q.s. ad 100% |

One example embodiment of an amitraz solution includes 0.100% w/w of Amitraz in 99.9% light mineral oil. Another example of an amitraz ointment can include 0.100% w/w of Amitraz and 29.9% mineral oil and 70.0% petrolatum.

To further illustrate some embodiments and advantages thereof, Table 2 below lists several non-limiting specific examples of topical isoxazoline parasiticide formulations for illustrative purposes only. All ingredients are listed as % w/w or grams/100 grams of preparation.

TABLE 2

| Ingredient | Solution 1 | Solution 2 | Solution 3 | Solution 4 |
|---|---|---|---|---|
| Isoxazoline parasiticide | 0.250 | 0.250 | 0.250 | 0.250 |
| (e.g., mivorilaner, | | | | |
| umifoxolaner, | | | | |
| esafoxolaner, tigolaner, | | | | |
| or modoflaner) | | | | |
| Benzalkonium Chloride | 0.0050 | 0.0050 | 0.10 | 0.10 |
| HPMC | 0 | 0.20 | 0 | 0.20 |
| Polyoxyl 35 Castor oil | 5.0 | 5.0 | 5.0 | 5.0 |
| Glycerin | 1.6 | 1.6 | 1.6 | 1.6 |
| Tromethamine (TRIS) | 0.364 | 0.364 | 0.364 | 0.364 |
| Hydrochloric acid | 0.0765 | 0.0765 | 0.0765 | 0.0765 |
| pH | 7.7 | 7.7 | 7.7 | 7.7 |
| Purified Water (or better) | q.s. ad | q.s. ad | q.s. ad | q.s. ad |
| | 100% | 100% | 100% | 100% |

To further illustrate some embodiments and advantages thereof, Table 2A below lists additional non-limiting examples of topical isoxazoline parasiticide formulations for illustrative purposes only. All ingredients are listed as % w/w or grams/100 grams of preparation.

TABLE 2A

| Ingredient | Solution 1 |
|---|---|
| Isoxazoline parasiticide (e.g., mivorilaner, | 0.03%-0.50% |
| umifoxolaner, esafoxolaner, tigolaner, or modoflaner) | |
| Benzalkonium Chloride or Lauralkonium Chloride | 0.0050 |
| HPMC | 0%-0.50% |
| Castor oil solubilizing agent (e.g., polyoxyl 35 castor | 2.5%-5.0% |
| oil) | |
| A polyol Glycerin or | 1.2%-2.0% |
| Sorbitol or mannitol or | 4.4%-5.0% |
| Xylitol or erythritol | 2.5%-3.0% |
| Tromethamine (TRIS) | 0.25%-0.50% |
| Hydrochloric acid | 0.05-0.20% |
| pH | 7.5-7.9 |
| Purified Water (or better) | q.s. ad 100% |

To further illustrate some embodiments and advantages thereof, Table 2B below lists additional non-limiting examples of topical isoxazoline parasiticide formulations for illustrative purposes only. All ingredients are listed as % w/w or grams/100 grams of preparation.

TABLE 2B

| Ingredient | Solution 1 |
|---|---|
| Isoxazoline parasiticide (e.g., mivorilaner, | 0.03%-0.50% |
| umifoxolaner, esafoxolaner, tigolaner, or modoflaner) | |
| Benzalkonium Chloride or Lauralkonium Chloride | 0% |
| HPMC | 0%-0.50% |
| Glycerin | 1.1-1.2% |
| Castor oil solubilizing agent (e.g., polyoxyl 35 castor | 2.5%-5.0% |
| oil) | |
| Na₂HPO₄•7H₂O, | 0.020M |
| Na₂HPO₄•H₂O | |
| Potassium Sorbate | ≤0.30% |
| Edetate Disodium | 0.020% |
| pH | 7.2 |
| Purified Water (or better) | q.s. ad 100% |

To further illustrate some embodiments and advantages thereof, Table 3 below lists several non-limiting specific examples of topical gel isoxazoline parasiticide formulations for illustrative purposes only. All ingredients are listed as % w/w or grams/100 grams of preparation.

TABLE 3

| Ingredient | Formulation 1 | Formulation 2 | Formulation 3 | Formulation 4 |
|---|---|---|---|---|
| Isoxazoline parasiticide (e.g., | 0.250 | 0.250 | 0.250 | 0.250 |
| mivorilaner, umifoxolaner, | | | | |
| esafoxolaner, tigolaner, or | | | | |
| modoflaner) | | | | |
| Cremophor EL | 5.0 | 5.0 | 5.0 | 5.0 |
| HPMC | 0.20 | 0.20 | 0.20 | 0.20 |
| Carbomer 974P | 0.50 | 0.80 | 0 | 0 |
| Carbomer TR1 | 0 | 0 | 0.50 | 0.80 |
| Glycerin | 1.7 | 1.7 | 1.7 | 1.7 |
| TRIS | pH adjusted | pH adjusted | pH adjusted | pH adjusted |
| Lauralkonium Chloride | 0.0075 | 0.0075 | 0.0075 | 0.0075 |
| NaH₂PO₄7H₂O | 0.429 | 0.429 | 0.429 | 0.429 |
| Na₂HPO₄ | 0.0456 | 0.0456 | 0.0456 | 0.0456 |
| pH | 7.25 | 7.25 | 7.25 | 7.25 |
| Purified Water (or better) | q.s. ad 100% | q.s. ad 100% | q.s. ad 100% | q.s. ad 100% |

To further illustrate some embodiments and advantages thereof, Table 4 below lists several non-limiting specific examples of topical gel isoxazoline parasiticide formulations for illustrative purposes only. All ingredients are listed as % w/w or grams/100 grams of preparation.

TABLE 4

| Ingredient | Formulation 5 | Formulation 6 | Formulation 7 | Formulation 8 |
|---|---|---|---|---|
| Isoxazoline parasiticide (e.g., | 0.250 | 0.250 | 0.250 | 0.250 |
| mivorilaner, umifoxolaner, | | | | |
| esafoxolaner, tigolaner, or | | | | |
| modoflaner) | | | | |
| Cremophor EL | 5.0 | 5.0 | 5.0 | 5.0 |
| HPMC | 0.20 | 0.20 | 0.20 | 0.20 |
| Carbomer 974P | 0.50 | 0.80 | 0 | 0 |
| Carbomer TR1 | 0 | 0 | 0.50 | 0.80 |
| Glycerin | 1.7 | 1.7 | 1.7 | 1.7 |
| TRIS | pH adjusted | pH adjusted | pH adjusted | pH adjusted |
| Lauralkonium Chloride | 0.0075 | 0.0075 | 0.0075 | 0.0075 |
| NaH₂PO₄7H₂O | 0 | 0 | 0 | 0 |
| Na₂HPO₄ | 0 | 0 | 0 | 0 |
| pH | 7.25 | 7.25 | 7.25 | 7.25 |
| Purified Water (or better) | q.s. ad 100% | q.s. ad 100% | q.s. ad 100% | q.s. ad 100% |

In some embodiments, a topical ophthalmic formulation can include the following instructions for use. A patient can be instructed to shower or bathe first before applying the study medication and wash their hands before applying the study medication. A unit dose, such as a single drop of the formulation can be directly applied into each eye once or twice a day, e.g., once in the morning and once in the evening. After delivering the drop to the conjunctiva and/or cornea of the eye, the patient can close their eyes and apply gentle pressure to the upper lid to express the medication across their upper and lower eyelid margins. The formulation can then be allowed to dry without dabbing with a tissue. The formulation can then be stored at room temperature in a climate-controlled environment (15 to 30° C.), avoiding extreme heat or cold. In some embodiments, the patient is instructed not to apply any other topical ophthalmic medications within a specified period, e.g., one hour before and one hour after administering the study medication.

In some embodiments, systems and methods include qualitative and/or quantitative assessment of Demodex on an anatomical location of the patient, such as, for example, on eyelashes and/or within glands. In some embodiments, a method can include receiving a first assessment of a quantity of Demodex mites on an anatomical structure of the patient and initiating topical administration of the dermatologic and/or ophthalmic composition if the quantity of Demodex mites is greater than a predetermined value, such as greater than about 1, 1.5, 2, 2.5, 3, 4, 5, or more mites per square centimeter of skin (or mites per lash). In some embodiments, a method can include receiving a second assessment of a quantity of Demodex mites following therapy to quantitatively assess improvement, and either continuing, modifying (via an increase or decrease in dose, frequency, formulation, and the like), or discontinuing therapy based on the second assessment, which can be about, no more than about, or at least about 1 day, 2 days, 3 days, 5 days, 7 days, 10 days, 14 days, 21 days, 28 days, 30 days, or more or less after the first assessment. In some embodiments, the therapy results in a reduction of about or at least about 10%, 20%, 30%, 40%, 50%, 60%, 70%, 80%, 90%, or 100% eradication of the Demodex at the anatomic location.

In some embodiments, the formulation or method can result in a surprisingly durable effect, with no or substantially no recurrence or increase in the presence of Demodex mites for at least about 1, 2, 3, 4, or more weeks, or at least about 1, 2, 3, 4, 5, 6, 7, 8, 9, 10, 11, 12 months, or more after the last dosing to the patient.

The presence of cylindrical dandruff, also known as cylindrical casts, are scales that form clear cuffs collaring the eyelash root, at the base of the eyelash. Cylindrical dandruff on an eyelash is generally considered pathognomonic for Demodex infestation, can be diagnosed via epilation and viewed under a slit lamp microscope, and then counted automatically or manually. Skin surface biopsy (SSB) technique with cyanoacrylic adhesion is a commonly used method to measure the density of Demodex. It allows the collection of the superficial part of the horny layer and the contents of the pilosebaceous follicle. Other sampling methods used in assessing the presence of Demodex by microscopy include adhesive bands, skin scrapings, skin impressions, expressed follicular contents, comedone extraction, hair epilation, and punch biopsies.

In some embodiments, systems and methods for detecting Demodex in a subject are disclosed that do not necessarily require epilation. Such measures of diagnosis of Demodex can be advantageous because Demodex, particularly Demodex brevis can be challenging to detect and quantify via epilation. Furthermore, many patients object to epilation due to discomfort. Furthermore, initiation of treatment could be earlier and based on objective criteria.

For example, a device, such as a disposable hydrogel contact lens can be utilized to collect tears from a subject. This device, e.g., lens is then sent to a laboratory for detecting, and potentially quantifying, Demodex DNA by PCR or other means. The genome for both Demodex folliculorum and Demodex brevis have been sequenced. A “diagnostic” lens can be placed on the eye and removed after at a short fixed period, such as, for example, about or less than about 30, 20, 15, 10, or 5 minutes. Such a lens can made of a hydrogel with relatively high affinity for a Demodex biomarker, including DNA.

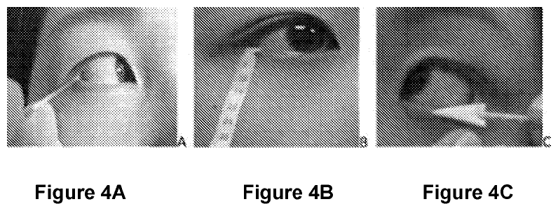

In some embodiments, tear sampling can utilize devices including capillary glass tubes to harvest tears from the lower lid tear meniscus as shown in FIG. 4A. This method can be especially useful when quantitative, small volumes are needed. In addition, evaporation can be eliminated, if beneficial to do so, simply by sealing both ends of the tube.

Some embodiments also include non-contact lens skin and tear sampling methods, such as, for example, a “litmus paper” or wicking paper embodiment similar to a Schirmer test (illustrated in FIG. 4B), or a lash brush harvesting technique (illustrated in FIG. 4C). In some embodiments, chitin, chitosan, or other Demodex-specific biomarkers that could be detected and quantified to correlate with mite numbers.

Demodex DNA can be quantified, for example, as the density of DNA copies coding for a particular Demodex target sequence (e.g., 18S rRNA as one non-limiting example). In some embodiments, a density (defined as the number of DNA copies coding for a target region of Demodex per ng of human gDNA (×10⁻⁶) of Demodex) can be a threshold to initiate therapy if greater than about 1, 2, 3, 4, 5, 6, 7, 8, 9, 10, 11, 12, 13, 14, 15, 16, 17, 18, 19, 20, or more.

An infestation could be further categorized as to causative species of Demodex (e.g., Demodex folliculorum vs Demodex brevis). Demodex brevis resides mostly within the Meibomian and sebaceous glands. Treatment could be modified, enhanced, or targeted based on the dominant species, e.g., increasing delivery of the therapeutic formulation to selected glands, for example.

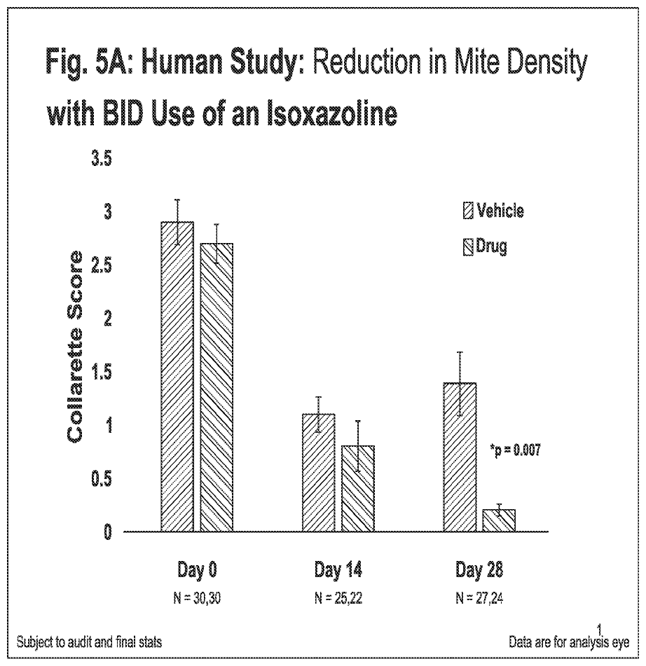

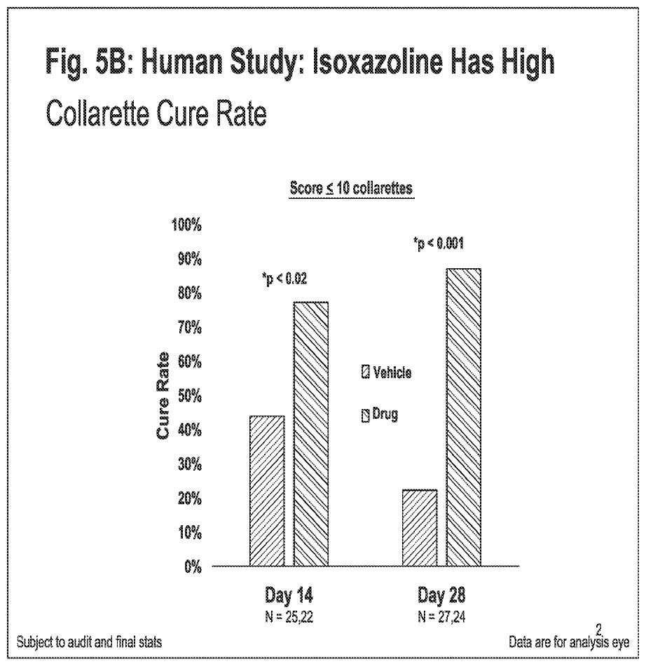

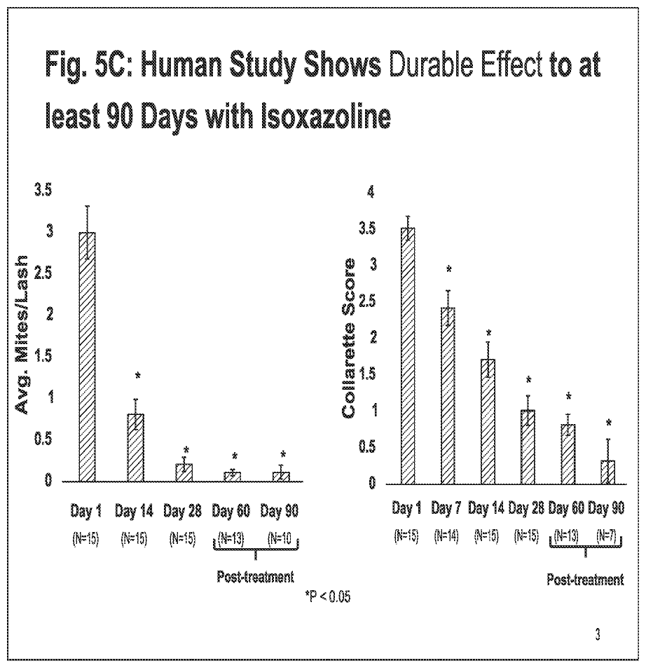

FIGS. 5A-5C illustrate human study data relating to a randomized clinical trial of an isoxazoline parasiticide eye drop formulation compared to vehicle, in patients with Demodex blepharitis. As illustrated in FIG. 5A, the collarette score of patients receiving the isoxazoline parasiticide formulation decreased dramatically, and statistically significantly compared to vehicle after 28 days of twice daily administration. As illustrated in FIG. 5B, the collarette cure rate, e.g., a score of less than or equal to 10 collarettes, was over 80% for the isoxazoline parasiticide formulation, and statistically significantly compared to vehicle after 28 days of twice daily administration. As illustrated in FIG. 5C, there was a durable effect after discontinuation of the isoxazoline parasiticide formulation after 28 days, with no rebound in number of mites per lash or collarette score at 60 days or 90 days, which was statistically significant. The aforementioned data illustrates unexpectedly rapid, complete, and durable responses of formulations and methods as disclosed herein never before seen in blepharitis.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “applying an isoxazoline parasiticide to an eye” includes “instructing the applying an isoxazoline parasiticide to an eye.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately,” “about,” and “substantially” as used herein include the recited numbers (e.g., about 10%=10%), and also represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of the stated amount.

What is claimed is:

1. A method for treating blepharitis in a patient, comprising:
topically administering directly to an ocular surface of one or more eyes of a patient in need of treatment thereof an effective amount of an isoxazoline parasiticide, formulated into an ophthalmic composition, the ophthalmic composition further comprising a pharmaceutically acceptable vehicle,
wherein the ophthalmic composition is sterile and non-irritating to the eye,
wherein the isoxazoline parasiticide is the sole active ingredient of the ophthalmic composition,
wherein the composition comprises a sorbate.

2. The method of claim 1, wherein the sorbate is the only preservative agent of the composition.

3. The method of claim 1, wherein the sorbate comprises between 0.01% and 0.50% by weight of the composition.

4. The method of any of claims 1-3, wherein the sorbate further comprises a chelating agent.

5. The method of any of claims 1-4, wherein the chelating agent comprises EDTA or edetate disodium.

6. The method of any of the preceding claims, wherein the ophthalmic composition comprises an eye drop.

7. The method of any of the preceding claims, wherein the ophthalmic composition does not include any essential oils.

8. The method of any of the preceding claims, wherein the isoxazoline parasiticide is selected from the group consisting of: fluralaner, sarolaner, lotilaner, afoxolaner, fluxametamide, isocycloseram, mivorilaner, umifoxolaner, esafoxolaner, tigolaner, and modoflaner.

9. The method of any of the preceding claims, wherein the ocular surface comprises at least one of the conjunctiva or cornea of the one or more eyes of the patient.

10. The method of any of the preceding claims, wherein the sorbate comprises potassium sorbate.

11. The method of any of the preceding claims, wherein the ophthalmic composition comprises a castor oil solubilizing agent, but not a polysorbate.

12. A method for treating blepharitis in a patient, comprising:
topically administering directly to one or more of the eye, eyelids, or eyelashes of a patient in need of treatment thereof an effective amount of an isoxazoline parasiticide, formulated into an ophthalmic composition further comprising a pharmaceutically acceptable vehicle, wherein the ophthalmic composition is sterile and non-irritating to the eye,
wherein the isoxazoline parasiticide is the sole active ingredient of the ophthalmic composition,
wherein the composition comprises a sorbate, and wherein the composition further comprises a castor oil solubilizing agent.

13. The method of claim 12, wherein the patient's eyes are closed upon topically administering the ophthalmic composition, such that the composition contacts orifices of Meibomian glands of the patient and outside of eyelid margins of the patient.

14. The method of claim 13, further comprising spreading the composition onto the eyelashes and follicles of the eyelashes.

15. The method of claim 13, further comprising spreading the composition onto the eyelashes and follicles of the eyelashes with an applicator.

16. The method of claims 12-15, wherein from about 0.001% to about 1% of the isoxazoline parasiticide is administered.

17. The method of claims 12-16, comprising topically administering the ophthalmic composition at least once daily for at least about 2 weeks.

18. The method of claims 12-17, comprising topically administering the ophthalmic composition at least once daily for at least about 4 weeks.

19. A method for treating an ocular Demodex infestation in a patient, comprising:
topically administering directly to one or more of the eyes, eyelids, or eyelashes of one or more eyes of a patient in need of treatment thereof, an effective amount of an isoxazoline parasiticide, formulated into an ophthalmic composition further comprising a pharmaceutically acceptable vehicle,
wherein the ophthalmic composition is sterile and non-irritating to the eye,
wherein the isoxazoline parasiticide is the sole active ingredient of the ophthalmic composition,
wherein the ophthalmic composition comprises a sorbate, wherein a volume of the composition delivered is less than about 100 microliters.

20. The method of claim 19, further comprising receiving a first assessment of a quantity of Demodex mites on an anatomical structure of the patient, and topically administering the ophthalmic composition if the quantity of Demodex mites is greater than a predetermined value.

21. The method of claims 19-20, further comprising receiving a first assessment of a quantity of collarettes (cylindrical dandruff) on an anatomical structure of the patient, and topically administering the ophthalmic composition if the quantity of collarettes is greater than a predetermined value.

22. The method of claims 19-21, wherein the ophthalmic formulation causes an abdomen and tail of Demodex mites on the patient to stop moving more quickly relative to a cephalothorax of the Demodex mites.

23. A method of treating blepharitis and/or rosacea, comprising:
topically applying a composition comprising isoxazoline parasiticide proximate one or more eyelashes, the topically applying therapeutically effective to preferentially be absorbed by a body of the Demodex mite with respect to ingestion by the Demodex mite sufficient to cause reduced movement of the body of the Demodex mite with respect to a head of the Demodex mite, the method sufficient to reduce or eliminate Demodex mites proximate the eyelashes, resulting in improvement of the manifestations of blepharitis and/or rosacea,
wherein the composition further comprises a sorbate and a castor oil solubilizing agent, wherein the sorbate is the sole preservative agent of the composition.

24. A topical ophthalmic formulation for treating blepharitis in a patient, comprising:
an effective amount of an isoxazoline parasiticide and a pharmaceutically acceptable vehicle,
wherein the ophthalmic composition is sterile and non-irritating to the eye,
wherein the isoxazoline parasiticide is the sole active ingredient of the ophthalmic composition,
wherein the ophthalmic composition further comprises a sorbate and a castor oil solubilizing agent, wherein the sorbate is the sole preservative agent of the composition.

25. The topical ophthalmic formulation of claim 24, wherein from about 0.01% to about 1% of the isoxazoline parasiticide with respect to the total weight of the composition is administered.

26. The topical ophthalmic formulation of claim 24, wherein about 0.03% by weight of the isoxazoline parasiticide with respect to the total weight of the composition is administered.

27. The topical ophthalmic formulation of claim 24, wherein about 0.10% by weight of the isoxazoline parasiticide with respect to the total weight of the composition is administered.

28. The topical ophthalmic formulation of claim 24, wherein about 0.30% by weight of the isoxazoline parasiticide with respect to the total weight of the composition is administered.

29. The topical ophthalmic formulation of claims 24-28, wherein the ophthalmic composition comprises an eye drop.

30. The topical ophthalmic formulation of claims 24-29, wherein the ophthalmic composition does not include any essential oils.

31. The topical ophthalmic formulation of claims 24-30, wherein the isoxazoline parasiticide is selected from the group consisting of: fluralaner, sarolaner, lotilaner, afoxolaner, fluxametamide, isocycloseram, mivorilaner, umifoxolaner, esafoxolaner, tigolaner, and modoflaner.

32. A topical formulation for use in treating an ocular surface disease, comprising:
an isoxazoline parasiticide;
at least one of Pemulen and HPMC;
a castor oil solubilizing agent;
glycerin;
a buffering agent; and
potassium sorbate,
wherein the formulation is therapeutically effective to reduce or eliminate Demodex mites proximate the eyelashes, resulting in improvement of the manifestations of blepharitis and/or rosacea.

33. The topical formulation of claim 32, for use in treating blepharitis.

34. The topical formulation of claim 32, for use in treating anterior blepharitis.

35. The topical formulation of claim 32, for use in treating posterior blepharitis.

36. The topical formulation of claims 32-35, for use in treating ocular rosacea.

37. A method for treating lice, scabies, and/or bed bugs in a patient, comprising:
topically administering directly to a skin surface of a patient in need of treatment thereof an effective amount of an isoxazoline parasiticide, formulated into a composition, the composition further comprising a pharmaceutically acceptable vehicle,
wherein the composition is non-irritating to the skin,
wherein the isoxazoline parasiticide is the sole active ingredient of the composition.

38. A method for preventing vector-borne disease in a patient, comprising:
topically administering directly to a skin surface of a patient in need of treatment thereof an effective amount of an isoxazoline parasiticide, formulated into a composition, the composition further comprising a pharmaceutically acceptable vehicle,
wherein the composition is non-irritating to the skin,
wherein the isoxazoline parasiticide is the sole active ingredient of the composition.

39. A method for improving eyelash health in a patient, comprising:
topically administering directly to an ocular surface of one or more eyes of a patient in need of treatment thereof an effective amount of an isoxazoline parasiticide, formulated into an ophthalmic composition, the ophthalmic composition further comprising a pharmaceutically acceptable vehicle,
wherein the ophthalmic composition is sterile and non-irritating to the eye,
wherein the isoxazoline parasiticide is the sole active ingredient of the ophthalmic composition,
wherein improving eyelash health increases the number, growth rate, thickness, and/or appearance of eyelashes of the patient.

40. The method of claim 39, wherein the composition is not systemically absorbed sufficient to cause a therapeutic systemic effect.

41. A topical, multi-dose ophthalmic formulation for use in treating an ocular surface disease, comprising:
an isoxazoline parasiticide;
a castor oil solubilizing agent;
a polyol;
an acid;
tromethamine (TRIS); and
lauralkonium or benzalkonium chloride,
wherein the formulation is therapeutically effective to reduce or eliminate Demodex mites proximate the eyelashes, resulting in improvement of the manifestations of blepharitis and/or rosacea.

42. The topical formulation of claim 41, for use in treating blepharitis.

43. The topical formulation of claim 41, for use in treating anterior blepharitis.

44. The topical formulation of claim 41, for use in treating posterior blepharitis.

45. The topical formulation of claims 41-44, for use in treating ocular rosacea.

46. The topical ophthalmic formulation of claims 41-45, wherein the isoxazoline parasiticide comprises between about 0.01% and about 0.50% w/w of the formulation.

47. The topical ophthalmic formulation of claims 41-45, wherein the isoxazoline parasiticide comprises about 0.10% w/w of the formulation.

48. The topical ophthalmic formulation of claims 41-45, wherein the isoxazoline parasiticide comprises about 0.30% w/w of the formulation.

49. The topical ophthalmic formulation of claims 41-48, wherein the ophthalmic composition does not include any essential oils.

50. The topical ophthalmic formulation of claims 41-49, wherein the isoxazoline parasiticide is selected from the group consisting of: fluralaner, sarolaner, lotilaner, afoxolaner, fluxametamide, isocycloseram, mivorilaner, umifoxolaner, esafoxolaner, tigolaner, and modoflancr.

51. The topical ophthalmic formulation of claims 41-50, wherein the castor oil solubilizing agent comprises between about 3% and about 7% w/w of the formulation.

52. The topical ophthalmic formulation of claims 41-51, wherein the castor oil solubilizing agent comprises about 5% w/w of the formulation.

53. The topical ophthalmic formulation of claims 41-52, wherein the polyol is selected from the group consisting of glycerol, xylitol, erythritol, mannitol, and sorbitol.

54. The topical ophthalmic formulation of claims 41-53, wherein the formulation further comprises HPMC or CMC.

55. The topical ophthalmic formulation of claims 41-54, wherein the formulation does not comprise a phosphate buffer.

56. The topical ophthalmic formulation of claims 41-55, wherein the formulation does not comprise a polysorbate.

57. The topical ophthalmic formulation of claims 41-56, wherein the formulation results only in a local therapeutic effects without any systemic effects.

58. A topical, single-dose ophthalmic formulation for use in treating an ocular surface disease, comprising:
an isoxazoline parasiticide;
a castor oil solubilizing agent;
a polyol; and
tromethamine (TRIS);
wherein the formulation does not comprise a preservative agent,
wherein the formulation is therapeutically effective to reduce or eliminate Demodex mites proximate the eyelashes, resulting in improvement of the manifestations of blepharitis and/or rosacea.

59. The topical ophthalmic formulation of claim 58, wherein the formulation results only in a local therapeutic effects without any systemic effects.

60. A topical, multi-dose ophthalmic formulation for use in treating an ocular surface disease, comprising:
an isoxazoline parasiticide;
a castor oil solubilizing agent;
a viscosity-enhancing agent;
a buffering agent;
a sorbate; and
a chelating agent,
wherein the formulation is therapeutically effective to reduce or eliminate Demodex mites proximate the eyelashes, resulting in improvement of the manifestations of blepharitis and/or rosacea.

61. The formulation of claim 60, wherein the sorbate comprises potassium sorbate, the chelating agent comprises EDTA or a salt of EDTA, and the viscosity-enhancing agent comprises HPMC.

62. The formulation of claim 60 or 61, wherein the pH of the formulation is between about 7 and about 7.5.

63. The formulation of claims 60-62, wherein the pH of the formulation is about 7.2.

64. A topical, ophthalmic formulation for use in treating an ocular surface disease, comprising:
an isoxazoline parasiticide;
a castor oil solubilizing agent; and
a polyol;
wherein the formulation is therapeutically effective to reduce or eliminate Demodex mites proximate the eyelashes, resulting in improvement of the manifestations of blepharitis and/or rosacea.

65. The topical formulation of claim 64, for use in treating blepharitis.

66. The topical formulation of claim 64, for use in treating anterior blepharitis.

67. The topical formulation of claim 64, for use in treating posterior blepharitis.

68. The topical formulation of claims 64-67, for use in treating ocular rosacea.

69. The topical ophthalmic formulation of claims 64-68, wherein the isoxazoline parasiticide comprises between about 0.01% and about 0.50% w/w of the formulation.

70. The topical ophthalmic formulation of claims 64-68, wherein the formulation is an eye drop formulation.

71. The topical ophthalmic formulation of claims 64-68, wherein the isoxazoline parasiticide comprises about 0.30% w/w of the formulation.

72. The topical ophthalmic formulation of claims 64-71, wherein the ophthalmic composition does not include any essential oils.

73. The topical ophthalmic formulation of claims 64-72, wherein the isoxazoline parasiticide is selected from the group consisting of: fluralaner, sarolaner, lotilaner, afoxolaner, fluxametamide, isocycloseram, mivorilaner, umifoxolaner, esafoxolaner, tigolaner, and modoflaner.

74. The topical ophthalmic formulation of claims 64-73, wherein the castor oil solubilizing agent comprises between about 3% and about 7% w/w of the formulation.

75. The topical ophthalmic formulation of claims 64-74, wherein the castor oil solubilizing agent comprises about 5% w/w of the formulation.

76. The topical ophthalmic formulation of claims 64-75, wherein the polyol is selected from the group consisting of glycerol, xylitol, erythritol, mannitol, and sorbitol.

77. The topical ophthalmic formulation of claims 64-76, wherein the formulation further comprises HPMC or CMC.

78. The topical ophthalmic formulation of claims 64-77, wherein the formulation does not comprise a phosphate buffer.

79. The topical ophthalmic formulation of claims 64-78, wherein the formulation does not comprise a polysorbate.

80. The topical ophthalmic formulation of claims 64-79, wherein the formulation results only in a local therapeutic effects without any systemic effects.
